(19) United States
(12) Patent Application Publication
(10) Pub. No.: US 2022/0313098 A1
(43) Pub. Date: Oct. 6, 2022

(54) WEARABLE BIOMETRIC WAVEFORM ANALYSIS SYSTEMS AND METHODS

(71) Applicant: VALENCELL, INC., Raleigh, NC (US)

(72) Inventors: Steven Francis LeBoeuf, Raleigh, NC (US); Tushar Dilip Tank, Raleigh, NC (US)

(21) Appl. No.: 17/635,613

(22) PCT Filed: Sep. 3, 2020
(86) PCT No.: PCT/US2020/049229

§ 371 Date: Feb. 15, 2022

Related U.S. Application Data
(60) Provisional application No. 62/896,836, filed on Sep. 6, 2019.

(51) Int. Cl. A61B 5/0205; A61B 5/318; A61B 7/00; A61B 5/11; A61B 5/00

(52) U.S. Cl. CPC A61B 5/0205; A61B 5/318; A61B 7/003; A61B 5/11; A61B 5/0077; A61B 5/6801; A61B 5/7275; A61B 5/7221; A61B 5/7264; A61B 2562/0204; A61B 5/02416

(57) ABSTRACT

A biometric waveform analysis system includes a wearable device having a sensor system and that is configured to be worn on a body of a subject, a metric output generator in communication with the sensor system, a waveform analysis engine in communication with the sensor system, and a control processor configured to control the sensor system, the metric output generator, and the waveform analysis engine. The sensor system includes one or more physiological sensors and motion sensors. The metric output generator includes logic that extracts at least one physiological parameter from the physiological data signal and extracts at least one motion parameter from the motion data signal. The waveform analysis engine includes waveform capture logic, normalization logic, contextual logic, and physiological assessment logic. The waveform capture logic receives the physiological data signal from the sensor system and separates the physiological data signal into a plurality of individual physiological waveforms.

### RELATED APPLICATIONS

This application is a 35 U.S.C. § 371 national stage application of PCT Application No. PCT/US2020/049229, filed on Sep. 3, 2020, which claims the benefit of and priority to U.S. Provisional Patent Application No. 62/896,836 filed Sep. 6, 2019, the disclosures of which are incorporated herein by reference as if set forth in their entireties. The above-referenced PCT International Application was published in the English language as International Publication No. WO 2021/046237 A1 on Mar. 11, 2021.

### FIELD OF THE INVENTION

The present invention relates generally to wearable devices, and more particularly to wearable biometric sensor technology for physiological monitoring for medical, health, and fitness applications.

### BACKGROUND OF THE INVENTION

Wearable devices used today are capable of assessing various biometrics from users throughout daily life activity, with varying levels of accuracy. However, the devices used today are at best only able to measure a handful of biometrics per device and are not capable of generating numerous physiological assessments from a single wearable device. Thus, users must wear multiple devices having various different sensing modalities and form-factors in order to generate a sufficient number of biometrics and to generate numerous meaningful physiological assessments. Moreover, wearable biometric sensing devices used today have not been able to effectively handle real-time processing and post-processing within the same wearable device, and are thus incapable of effectively measuring a plurality of biometrics in real-time while simultaneously generating numerous physiological assessments asynchronously over a period of time. Without this capability, wearable biometric sensing devices have been relegated to measuring biometrics in real time and to wirelessly send these processed biometrics to a remote device, having greater data storage capacity and greater processing power, in order to process physiological assessments. And because the systems required for measuring biometrics have been largely disparate from the systems required for generating physiological assessments from these biometrics, the development of smart wearable sensing solutions, requiring a tight feedback loop between measuring and assessing, has been stifled. Thus, the big vision of providing key actionable insights for improving one's health, as shown in FIG. 1, has not come to fruition.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

A biometric waveform analysis system includes a wearable device having a sensor system and configured to be worn on a body of a subject, at least one metric output generator in communication with the sensor system, at least one waveform analysis engine in communication with the sensor system, and at least one control processor configured to control the sensor system, the metric output generator, and the waveform analysis engine. The sensor system includes at least one physiological sensor and at least one motion sensor. The at least one physiological sensor is configured to sense physiological information from the subject and generate a physiological data signal that contains physiological waveform data. For example, in some embodiments, the at least one physiological sensor includes one or more of the following: a photoplethysmography (PPG) sensor, a bioimpedance sensor, an auscultatory sensor, a camera, an electrocardiogram (ECG) sensor, an electroencephalography (EEG) sensor, an electromyography (EMG) sensor, a body temperature sensor, and an electrooculography (EOG) sensor.

The at least one motion sensor is configured to obtain motion information from the subject and generate a motion data signal. Exemplary motion sensors that may be utilized include an inertial sensor, a piezoelectric sensor, an optical sensor, and a noise reference sensor.

The at least one metric output generator includes logic configured to extract at least one physiological parameter from the physiological data signal and to extract at least one motion parameter from the motion data signal.

The at least one waveform analysis engine includes waveform capture logic, normalization logic, contextual logic, and physiological assessment logic. The waveform capture logic is configured to receive the physiological data signal from the sensor system and separate the physiological data signal into a plurality of individual physiological waveforms. The normalization logic is configured to normalize the individual physiological waveforms. The contextual logic is configured to receive the motion data signal from the sensor system, identify subject physical activity information from the motion data signal, and tag the individual physiological waveforms with the subject physical activity information. The physiological assessment logic is configured to extract physiological information from the individual physiological waveforms and generate a physiological assessment of the subject based on the extracted physiological information. The physiological assessment of the subject may include one or more of the following: a blood flow assessment, a cardiac output assessment, a blood hydration assessment, a blood pressure assessment, an arrhythmia assessment, an assessment of subject stress.

In some embodiments, the sensor system also includes at least one environmental sensor configured to obtain environmental information in a vicinity of the subject, and the contextual logic is further configured to tag the individual physiological waveforms with the environmental information.

In some embodiments, the sensor system also includes preprocessing logic configured to filter the physiological data signal to provide cleaner physiological waveform data, i.e., physiological waveform data with reduced noise.

In some embodiments, the at least one metric output generator logic is further configured to generate a multiplexed serial data output of the extracted at least one physiological parameter and the extracted at least one motion parameter.

In some embodiments, the at least one waveform analysis engine further includes classifier logic configured to classify the individual physiological waveforms according to waveform type. The classifier logic may be configured to classify the individual physiological waveforms as normal or abnormal. The classifier logic may be configured to classify the individual physiological waveforms as one of a plurality of types of normal waveforms.

In some embodiments, the physiological assessment logic is configured to identify at least one characteristic feature of the individual physiological waveforms, and to generate the physiological assessment based on the identified at least one characteristic feature of the individual physiological waveforms. In some embodiments, the individual physiological waveforms are PPG waveforms, and the at least one characteristic is dicrotic notch information.

In some embodiments, the biometric waveform analysis system further includes relationship logic configured to generate a relationship between the extracted at least one physiological parameter and the individual physiological waveforms. The relationship logic may be further configured to generate a relationship between the extracted at least one motion parameter and the individual physiological waveforms.

In some embodiments, the wearable device includes at least one actuator configured to adjust stability of the wearable device relative to the subject body in response to the physiological assessment of the subject. The at least one actuator may be configured to adjust stability of the wearable device relative to the subject body in response to the at least one physiological parameter (e.g., subject heart rate, etc.) extracted by the metric output generator and/or the at least one motion parameter extracted by the metric output generator.

In some embodiments, the at least one physiological sensor includes an acoustic sensor that is configured to obtain auscultatory sounds from the body of the subject, and the wearable device further includes at least one actuator configured to cause the wearable device to create an acoustic seal with the body of the subject such that a cavity is created that is in acoustic communication with the acoustic sensor.

In some embodiments, the wearable device further includes an electromagnetic emitter configured to stimulate a region of the body of the subject with electromagnetic energy in response to the at least one physiological parameter extracted by the metric output generator. In some embodiments, the electromagnetic emitter is configured to stimulate a vagal nerve of the subject. In some embodiments, the electromagnetic emitter is configured to stimulate blood perfusion at the region of the body.

In some embodiments, the physiological assessment of the subject is generated by the physiological assessment logic in response to the at least one physiological parameter extracted by the at least one metric output generator and/or the at least one motion parameter extracted by the metric output generator. For example, if the at least one physiological parameter extracted by the at least one metric output generator is a heart rate value that is above or below a threshold, the physiological assessment of the subject generated by the physiological assessment logic is an assessment of subject blood pressure. In some embodiments, if the at least one physiological parameter extracted by the at least one metric output generator is a heart rate value that is above or below a threshold, the physiological assessment of the subject generated by the physiological assessment logic is an assessment of subject cardiac output or volumetric blood flow. In some embodiments, if the at least one motion parameter extracted by the at least one metric output generator is above or below a threshold, the physiological assessment of the subject generated by the physiological assessment logic is an assessment of subject blood pressure. In some embodiments, if the at least one motion parameter extracted by the at least one metric output generator is on one side of a threshold, the physiological assessment of the subject generated by the physiological assessment logic is an assessment of subject blood pressure based on subject pulse volume, and, if the at least one motion parameter extracted by the at least one metric output generator is on an opposite side of the threshold, the physiological assessment of the subject generated by the physiological assessment logic is an assessment of subject blood pressure based on a waveform parameterization.

In some embodiments the biometric waveform analysis system further includes logic configured to analyze the motion data signal and to make an estimation as to whether the subject has fallen down, and to generate an output parameter indicating the estimation. The physiological assessment then includes a determination that the subject has fallen down.

In some embodiments the biometric waveform analysis system further includes logic configured to analyze the physiological data signal and to make an estimation as to whether the subject has fallen down, and to generate an output parameter indicating the estimation. The physiological assessment then includes a determination that the subject has fallen down.

In some embodiments, the at least one physiological sensor includes a PPG sensor configured to emit and detect light at a plurality of distinct wavelengths, the waveform capture logic is configured to receive physiological data signals from the PPG sensor and separate the PPG data signals into a plurality of individual waveforms, and the physiological assessment logic is configured to identify at least one characteristic feature for at least two of the individual PPG waveforms, wherein the at least two of the PPG waveforms are associated with differing distinct electromagnetic wavelengths. The physiological assessment logic is configured to process a physiological assessment for the subject by processing the at least one characteristic feature from the at least two of the PPG waveforms.

According to other embodiments of the present invention, a method of generating a continuous physiological assessment for a subject, wherein the subject is wearing a PPG sensor that generates a PPG data signal comprising high rate PPG data and PPG waveform data, includes separating the PPG data signal into a plurality of individual PPG waveforms, generating at least one high-rate physiological metric from the PPG signal, generating a high-acuity physiological assessment of the subject from the PPG waveform data, over a time interval, by processing the PPG waveforms via at least one waveform analysis engine, wherein the at least one high-rate physiological metric is mapped to the high-acuity assessment over the time interval, and then outputting a continuous physiological assessment for the subject, wherein at least some physiological assessment values are based on a mapped relationship between the at least one high-rate physiological metric and the high-acuity physiological assessment.

In some embodiments, the high-acuity physiological assessment includes an assessment of blood pressure, the at least one high-rate physiological metric includes blood pulse volume information, and the continuous physiological assessment includes blood pressure information. In some embodiments, the high-acuity physiological assessment includes an assessment of cardiac output, the at least one high-rate physiological metric includes blood pulse volume information and heart rate information, and the continuous physiological assessment includes cardiac output information. In some embodiments, the high-acuity physiological assessment includes an assessment of volumetric blood flow, the at least one high-rate physiological metric includes blood pulse volume information, and the continuous physiological assessment includes volumetric blood flow information.

According to other embodiments of the present invention, a method of processing PPG waveforms generated by a PPG sensor worn on a body of a subject includes obtaining a PPG waveform from a PPG data signal from the PPG sensor, classifying the PPG waveform according to PPG waveform type, and processing the PPG waveform using a processing method selected based on the waveform type of the PPG waveform. Processing the PPG waveform using the processing method selected based on the waveform type may include generating a physiological assessment of the subject, such as a blood pressure assessment. The classifying step may include classifying the PPG waveform as a normal PPG waveform or as an abnormal PPG waveform. The classifying step may include classifying the PPG waveform as one of a plurality of types of normal PPG waveforms.

According to other embodiments of the present invention, a method of processing PPG waveforms generated by a PPG sensor worn on a body of a subject includes obtaining a PPG data signal from the PPG sensor, separating the PPG data signal into a plurality of individual PPG waveforms, classifying the individual PPG waveforms according to waveform type, and storing the classified individual PPG waveforms. Classifying the individual PPG waveforms may include classifying the individual PPG waveforms as normal or abnormal. Classifying the individual PPG waveforms may include classifying the individual PPG waveforms as one of a plurality of types of normal waveforms.

According to other embodiments of the present invention, a method of generating a physiological assessment of a subject includes obtaining a PPG data signal from a PPG sensor worn on a body of the subject, separating the PPG data signal into a plurality of individual PPG waveforms, buffering the PPG waveforms, classifying the buffered PPG waveforms into respective categories according to a physiological parameter associated with the subject and/or a temporal parameter associated with the subject, processing at least two PPG waveforms within each category to generate a respective representative PPG waveform in each category, and processing the representative PPG waveform in at least two of the categories to generate a physiological assessment for the subject. Exemplary physiological assessments include a blood pressure assessment, an assessment of subject stress, a cardiac assessment, a pulmonary assessment, a cardiopulmonary assessment, and a circadian assessment.

In some embodiments, processing the representative PPG waveform in at least two of the categories to generate the physiological assessment of the subject further includes processing food diary information to determine a correlation between the average PPG waveform in each of the at least two of the categories with a diet of the subject.

In some embodiments, the method of generating a physiological assessment of a subject further includes creating a historical record of the representative PPG waves, and processing the historical record to generate a historical physiological assessment of the subject.

According to other embodiments of the present invention, methods of determining whether a subject wearing a sensor device has fallen down are provided. The sensor device has at least one physiological sensor configured to sense physiological information from the subject, at least one motion sensor configured to obtain motion information from the subject, and at least one processor. The method includes processing a motion data signal from the at least one motion sensor to determine if it is likely that the subject has fallen down, and in response to determining it is likely that the subject has fallen down, processing a physiological data signal from the at least one physiological sensor to confirm whether the subject has fallen down.

In some embodiments, the physiological sensor is a PPG sensor, and processing the physiological data signal includes processing subject heart rate information in the physiological data signal to determine that a heart rate of the subject has substantially changed. In some embodiments, the physiological sensor is an acoustic sensor, and processing the physiological data signal includes processing auscultatory information in the physiological data signal to determine that subject breathing has substantially changed. In some embodiments, the physiological sensor is a PPG sensor, and processing the physiological data signal includes processing subject blood pressure information in the physiological data signal to determine that subject blood pressure has substantially changed.

According to other embodiments of the present invention, a method of determining whether a subject wearing a senor device has fallen down is provided. The sensor device includes at least one acoustic sensor, at least one motion sensor configured to obtain motion information from the subject, and at least one processor. The method includes processing a motion data signal from the at least one motion sensor to determine if it is likely that the subject is falling down, and in response to determining it is likely that the subject is falling down, processing an acoustic data signal from the at least one acoustic sensor to identify a subject impact signal in order to confirm whether the subject has fallen down.

According to other embodiments of the present invention, a method of determining whether a subject wearing a sensor device has fallen down is provided. The sensor device includes at least one optical sensor, at least one motion sensor configured to obtain motion information from the subject, and at least one processor. The method includes processing a motion data signal from the at least one motion sensor to determine if it is likely that the subject is falling down, and in response to determining it is likely that the subject is falling down, processing a data signal from the at least one optical sensor to identify a subject impact signal in order to confirm whether the subject has fallen down.

According to other embodiments of the present invention, a PPG sensor includes an optical detector, a first set of optical emitters positioned around the optical detector, and a second set of optical emitters positioned around the first set of optical emitters in concentric relationship therewith. The first set of optical emitters is configured to generate at least one wavelength of light that is different from that of the second set of optical emitters.

According to other embodiments of the present invention, a PPG sensor includes an optical detector, a first optical emitter extending around the optical detector, and a second optical emitter radially spaced apart from the first optical emitter and extending around the first optical emitter. The first and second optical emitters are in concentric relationship with the optical detector. The first optical emitter emits light at a first wavelength, and the second optical emitter emits light at a second wavelength different from the first wavelength.

According to other embodiments of the present invention, a PPG sensor includes an optical detector, and a plurality of optical emitters positioned around the optical detector. A first set of the plurality of optical emitters are oriented to emit light in a first direction, and a second set of the plurality of optical emitters are oriented to emit light in a second direction. The first direction extends outwardly from a direction normal to a light receiving surface of the optical detector, and the second direction extends inwardly towards the direction normal to the light receiving surface of the optical detector. In some embodiments, the plurality of optical emitters is positioned around the optical detector in a substantially equidistant, circumferential spaced-apart relationship to define a plurality of pairs of opposing emitters, and each emitter in a pair is oriented in the same first or second direction.

According to other embodiments of the present invention, a method of generating a physiological assessment for a subject via a sensor device worn by the subject is provided. The sensor device includes a motion sensor, a PPG sensor, a tertiary sensor, and at least one processor. The method includes screening, via the at least one processor, a motion signal generated by the motion sensor to determine if PPG monitoring of the subject is warranted, in response to determining that PPG monitoring of the subject is warranted, screening, via the at least one processor, a PPG signal from the PPG sensor to determine if monitoring of the subject via the tertiary sensor is warranted, in response to determining that monitoring of the subject via the tertiary sensor is warranted, screening, via the at least one processor, a signal from the tertiary sensor, and processing, via the at least one processor, the signal from the tertiary sensor to generate a physiological assessment for the subject.

In some embodiments, the tertiary sensor is an ECG sensor and the tertiary signal is an ECG signal. In some embodiments, the tertiary sensor is an acoustic sensor and the tertiary signal is an auscultatory signal.

The physiological assessment may include one or more of the following: an assessment of a cardiac condition of the subject, an assessment of a presence of arrhythmia in the subject, an assessment of a breathing condition of the subject, an assessment that a cardiac condition is imminent, and an assessment that a respiratory condition is imminent.

According to other embodiments of the present invention, a method of generating a physiological assessment for a subject via a sensor device worn by the subject is provided. The sensor device includes a motion sensor, a PPG sensor, a tertiary sensor, and at least one processor. The method includes screening, via the at least one processor, a motion signal generated by the motion sensor to determine if PPG monitoring of the subject is warranted, in response to determining that PPG monitoring of the subject is warranted, screening, via the at least one processor, a PPG signal from the PPG sensor to determine if monitoring of the subject via the tertiary sensor is warranted, in response to determining that monitoring of the subject via the tertiary sensor is warranted, activating the tertiary sensor via the at least one processor, and processing, via the at least one processor, the motion signal, the PPG signal, and a signal from the tertiary sensor to generate a physiological assessment for the subject.

In some embodiments, the tertiary sensor is an ECG sensor, and activating the tertiary sensor includes activating the ECG sensor. In some embodiments, the tertiary sensor is an auscultatory sensor, and activating the tertiary sensor includes activating the auscultatory sensor. In some embodiments, the tertiary sensor is an optical sensor, and activating the tertiary sensor includes activating the optical sensor to emit light into the body of the subject. In some embodiments, the tertiary sensor is a camera sensor, and activating the tertiary sensor comprises activating the camera.

The physiological assessment may include an assessment of heart rate variably of the subject, an assessment of a cardiac or respiratory condition of the subject, etc.

According to other embodiments of the present invention, a method of generating a physiological assessment of a subject includes obtaining a photoplethysmography (PPG) data signal from a PPG sensor worn on a body of the subject, wherein the PPG data signal comprises a plurality of PPG waveforms, processing the PPG data signal to determine consistency of the PPG waveforms, classifying the PPG waveforms according to waveform consistency, and preferentially processing the PPG waveforms having a classification of higher consistency to generate a physiological assessment for the subject, such that the physiological assessment is more accurate than if all PPG waveforms had been utilized. In some embodiments, determining the consistency of the PPG waveforms comprises determining relative-peak-ratio (RPR) for the PPG waveforms. In some embodiments, the physiological assessment comprises an assessment of subject blood pressure.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate various embodiments of the present invention. The drawings and description together serve to fully explain embodiments of the present invention.

FIG. 1 illustrates an ideal vision of improving a person's health through the use of a smart wearable sensing device and a tight feedback loop between measurements taken by the device and personalized assessments made based upon those measurements.

FIG. 2 illustrates a wearable biometric waveform analysis system, according to some embodiments of the present invention.

FIG. 3 illustrates a specific embodiment of the wearable biometric waveform analysis system of FIG. 2.

FIG. 4 illustrates data from the data contextualizer of FIG. 3 serialized into a serial data stream.

FIGS. 5A-5C illustrate three classifications of PPG waveforms, respectively.

FIG. 6 illustrates a summary of known PPG waveform patterns.

FIG. 7 illustrates a PPG waveform processed by the waveform processor of FIG. 3, and which has been normalized in amplitude in the y-axis, with a peak amplitude set to “1”, and normalized in time in the x-axis, with a start point and stop point of ZC1 and ZC2, respectively.

FIGS. 8A-8B illustrate examples of waveform processing.

FIG. 9A illustrates eleven PPG waveforms taken from a subject wearing a PPG sensor.

FIG. 9B illustrates the spectral transform of the eleven waveforms of FIG. 9A, with a speak spectral amplitude at the heart rate of the subject (˜70 BPM).

FIGS. 10A-10B illustrate raw PPG waveform data taken from the same subject at the same body location using two distinct electromagnetic wavelengths in the green (˜520 nm) and IR (˜940 nm) ranges, showing a notable difference between the dicrotic notch (D) of each wavelength.

FIG. 11 illustrates a PPG waveform processed via the waveform processor of FIG. 3.

FIG. 12A is a bottom plan view of an exemplary wearable device having a sensor module disposed within a housing thereof for obtaining physiological information, such as heart rate, of a person wearing the device, according to some embodiments of the present invention.

FIG. 12B is a side view of the wearable device of FIG. 12A.

FIG. 12C is an exemplary wearable earpiece having a sensor module disposed therewithin for obtaining physiological information, such as heart rate, of a person wearing the device, according to some embodiments of the present invention.

FIGS. 13A-13C illustrate concentric ring optical configurations, according to some embodiments of the present invention.

FIGS. 14A-14C illustrate concentric ring optical configurations, according to some embodiments of the present invention.

FIGS. 15A-15B illustrate earpiece devices having a sensor module disposed within a housing thereof for monitoring the heart rate of a person wearing the device, and that include actuators according to some embodiments of the present invention.

FIG. 16A is a top plan view of an exemplary wearable device having a sensor module disposed within a housing thereof for monitoring the heart rate of a person wearing the device and also having an actuator, according to some embodiments of the present invention.

FIG. 16B is a bottom plan view of the device of FIG. 16A illustrating the sensor module.

FIG. 16C is a side view of the device of FIG. 16A.

FIG. 17A illustrates an earpiece device with multiple sensor regions, according to some embodiments of the present invention.

FIG. 17B illustrates an earpiece device with multiple sensor regions, according to some embodiments of the present invention.

FIG. 18 illustrates a wearable sensor device, according to some embodiments of the present invention.

FIG. 19 is a flowchart illustrating a method for improving the accuracy of a biometric or a physiological assessment, according to some embodiments of the present invention.

FIG. 20 illustrates exemplary equations for generating the autocorrelation representations of FIGS. 23 and 25.

FIG. 21 is a flowchart illustrating a method for improving the accuracy of a biometric or a physiological assessment, according to some embodiments of the present invention.

FIG. 22A illustrates PPG waveforms over a broad range.

FIG. 22B illustrates PPG waveforms over a narrow range.

FIG. 23 is a graphical representation of an autocorrelation of a set of PPG waveforms having a relatively high consistency (high-RPR), according to some embodiments of the present invention.

FIGS. 24A-24B illustrate an exemplary set of PPG waveforms collected during periodic breathing, according to some embodiments of the present invention.

FIG. 25 is a graphical representation of an autocorrelation of a set of PPG waveforms having a relatively low consistency (low-RPR), according to some embodiments of the present invention.

FIG. 26 illustrates a comparison of estimated (PPG-based) and measured (sphygmomanometer-based) blood pressure values, according to some embodiments of the present invention.

FIG. 27 illustrates a digit-worn apparatus, according to some embodiments of the present invention.

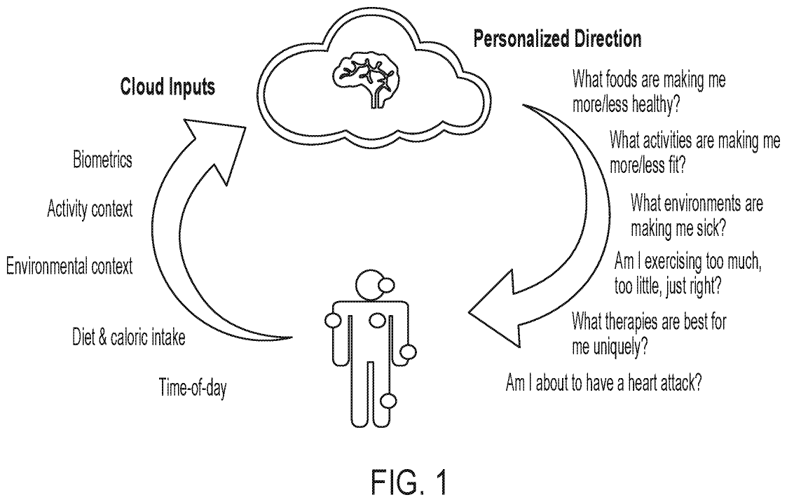

### DETAILED DESCRIPTION

As used herein, the term “processor” broadly refers to a signal processing circuit or computing system, or processing or computing method, which may be localized or distributed. As an example, a processing or computing method may be executed as computing instructions (or logic) in software or an algorithm. Alternatively, or additionally, the processor may comprise a physical circuit for processing analog or digital information. Microprocessors, microcontrollers, digital signal processing circuits, or analog signal processing circuits represent a few non-limiting examples of signal processing circuits (physical processors) that may be found in a localized and/or distributed system. A localized signal processing circuit may comprise one or more signal processing circuits or processing methods localized to a general location, such as to an activity monitoring device. Examples of such devices may comprise an earpiece, a headpiece, a finger clip, a toe clip, a limb band (such as an arm band or leg band), an ankle band, a wrist band, a nose band, a sensor patch, or the like. Examples of a distributed processing circuit comprise “the cloud,” the internet, a remote database, a remote processor computer, a plurality of remote processing circuits or computers in communication with each other, etc., or processing methods distributed amongst one or more of these elements. The key difference between the distributed and localized processing circuits is that a distributed processing circuit may include delocalized elements, whereas a localized processing circuit may work independently of a distributed processing system. As a nonlimiting example, a statement such “the system comprises an ‘X’ processor and a ‘Y’ processor” may refer to: 1) two differing processing methods or circuits, “X” and “Y”, operating within the same physical processor, 2) two differing processing methods or circuits, “X” and “Y”, operating in distinctly different physical processors, 3) two distinct physical processors “X” and “Y”, 4) two differing processing methods or circuits distributed over a plurality of distinct physical processors, or 5) a combination of one or more of the aforementioned. As a particular example, the statement “a control processor is configured to communicate between and control various processors” may mean that a piece of software code is configured to communicate between and control various other pieces of software code. Because, in this case, the software code is dedicated to controlling various processors, it is called a control processor. Alternatively, the statement may also mean that a microcontroller is configured to communicate between and control various other microcontrollers. These examples are nonlimiting to the meaning of the statement “a control processor is configured to communicate between and control various processors”, as various embodiments of processors, both physical and in software, are possible.

The term “real-time” is used herein to describe a process that requires a period of time that appears substantially real-time to a human individual. Thus, the term “real-time” is used interchangeably to mean “near real-time” or “quasi-real-time”. For example, in practicality, a process that requires less than 10 seconds to generate a heart rate metric for an individual may be considered to be real time, as used herein. In contrast, an assessment of cardiovascular fitness may require monitoring heart rate over a period of time that is several minutes long, or longer, which is not considered to be a real-time assessment.

The term “cleaner”, as used herein with reference to various different sensor signals, refers to cleaner, or “less noisy”, signals. For example, “cleaner physiological waveform data” refers to physiological waveforms that are cleaner, or less noisy, following a processing step, such as a filtering step or other processing step.

The term “high-rate”, as used herein with reference to the generation of biometrics or assessments, refers to “high throughput” biometrics or assessments that do not require intense processing resources or require long processing times. For example, a “high-rate” metric may comprise heart rate, processed via digital filtering on a second-by-second basis. In contrast, a “low-rate” metric may be blood pressure, processed via a complex machine learning model on a minute-by-minute basis. A high-rate metric or assessment takes less processing power or processing time than a low-rate metric or assessment, thereby making high-rate metrics and assessments more suitable for real-time processing.

The term “continuous”, as used herein with reference to physiological monitoring, refers to ongoing, autonomous monitoring at regular time intervals, without halting signal processing. In contrast, “intermittent” monitoring implies that signal processing is halted for a notable period of time. “Continuous” is a relative term, as “notable” is a relative term. Namely, the time span between measurements for continuous processing depends on the biometric or assessment being generated and is related to the likelihood of meaningful change. For example, as heart rate may significantly change on a second-by-second basis, a 1-second update for heart rate may be critical for continuous monitoring. In contrast, as white blood cell count may not significantly change over the course of several minutes, continuous monitoring of white blood cell count may be achieved via minute-by-minute monitoring, or even hourly monitoring.

The term “high acuity”, as used herein with respect to physiological monitoring, refers to “high-resolution”, “high-precision”, or “high-accuracy” monitoring. For example, a “high-acuity” assessment of blood pressure refers to an assessment of blood pressure that can be resolved within ±8.0 mmHg or less.

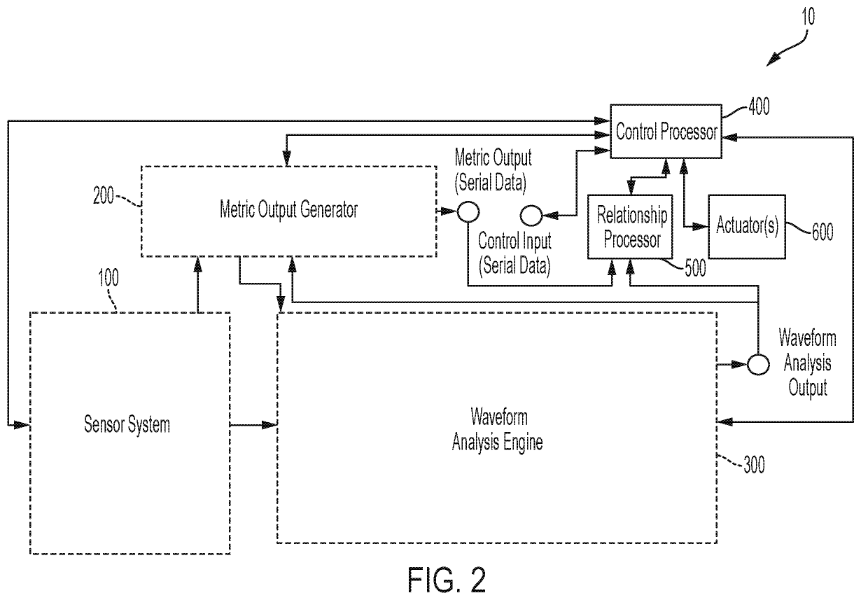

FIG. 2 summarizes the high-level invention for a wearable biometric waveform analysis system 10. A biometric waveform (also called a “physiological waveform”) is defined herein as a time-dependent waveform sensed from a living organism, such as that from optical, electrical, acoustic, or mechanical signals collected by at least one sensor. A definition of each key element of the system 10 is presented in Table 1 below:

TABLE 1
Definition of Key Elements of FIG. 2

| Element | Function |
|---|---|
| Sensor System | System comprising sensing and sensor preprocessing |
| 100 | elements. |
| Metric Output | Circuitry and/or algorithms for a) generating real-time or |
| Generator | quasi-real time metrics, such as various physiological and |
| 200 | motion parameters: i.e., artifact-reduced streaming PPG, |
| | heart rate, respiration rate, RR-interval, pulse wave |
| | volume, motion cadence, activity status, and the like and |
| | b) generating high-level assessments as needed. |
| Waveform | Processor configured to process and analyze waveforms |
| Analysis | received from the sensor system. |
| Engine | |
| 300 | |
| Control | Processor configured to communicate between and control |
| Processor | various processors in the invention. |
| 400 | |
| Relationship | Processor configured to generate at least one relationship |
| Processor | between an output from the Metric Output Generator and |
| 500 | an output from the Waveform Analysis Engine. |
| Actuator(s) | At least one actuator configured to actuate based on a |
| 600 | signal from the Control Processor. |

This invention effectively addresses a number of challenges that have not been sufficiently addressed in prior art. Namely, the invention:
1) Enables high-functionality with lower battery power.
2) Addresses the need for a single wearable sensor that can measure multiple biometrics and physiological assessments, some in real-time and some as a post assessment.
3) Enables a wearable biometric sensing system that can dynamically adapt to changing needs to focus resources where needed.
4) Enables a wearable biometric sensing system that can control the actuation of various body-worn actuators (which may help with further sensing).
5) Creates a means of normalizing sensor signal outputs from a wearable sensor so that generating new assessments is streamlined.
6) Creates a means of classifying PPG (photoplethysmography) waveforms into “families” such that they can be processed more effectively by “family”.
7) Enables a variety of compelling user experiences
8) Addresses the problem of universality in PPG sensing—accurately generating physiological assessments on a broad array of subjects having inherently different PPG characteristics.
9) Creates a means of normalizing biometric data inputs into the cloud for scalability across multiple platforms.

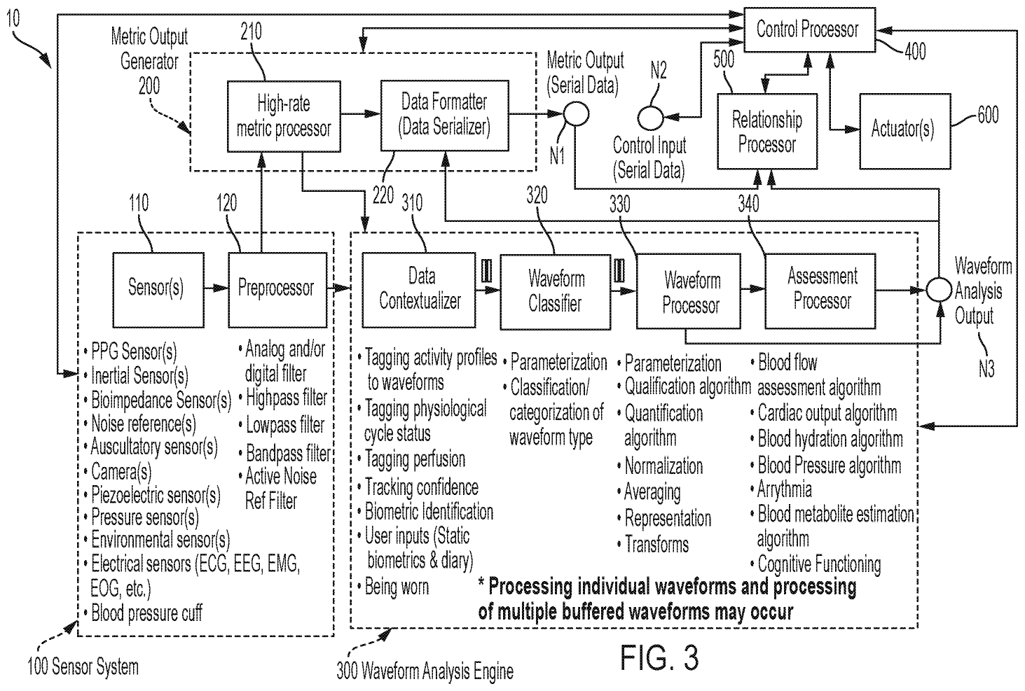

A specific embodiment of the wearable biometric waveform analysis system 10 of FIG. 2 is presented in FIG. 3. It should be noted that the arrows in FIG. 2 and FIG. 3 are meant to relate key communication channels between the various elements and subcomponents. The arrows do not necessarily imply a direct communication pathway; for example, other elements may in between the key elements shown. Rather, the arrows imply key communication channels that are important and especially noteworthy for at least one embodiment of the invention. Physically, the communication channel can be any suitable energy pathway, such as electrical, magnetic, mechanical, electromagnetic radiation, thermal, acoustic, and the like. A preferred communication channel would be an electrical channel (such as through circuitry) or digital (such as through software). It should also be noted that the parallel signs shown in FIG. 3, on each side of the waveform classifier 320, are presented simply to emphasize that the data contextualizer 310, waveform classifier 320, and waveform processor 330 may execute processing as series and/or parallel processing steps. However, this emphasis should not be misconstrued to imply that processing for other elements in the invention, not having such emphasis, must all be processed serially. It should also be noted that although memory storage is not explicitly shown in FIG. 2 or FIG. 3, electronic memory storage may be required for real-time data buffering and for long-term storage of data for post-processing. Typically, the assessment processor 340 may require the most frequent access to long-term memory storage, as assessments may be generated by processing several stored datasets.

A definition of each of the additional key elements of the preferred embodiment presented in FIG. 3 is listed in Table 2 below:

TABLE 2
Definition of Key Elements of FIG. 3

| Element | Function |
|---|---|
| Sensor(s) | Sensor elements configured to sense data for use in the |
| 110 | wearable biometric signal analysis system. |
| Preprocessor | Processor for filtering sensor data and generating cleaner |
| 120 | physiological waveforms of interest. |
| High-rate metric | Processor for generating real-time metrics and real-time |
| processor | assessments |
| 210 | |
| Data formatter | Processor for formatting the output of the high-rate metric |
| 220 | processor such that the real-time metrics and |
| | assessments can be communicated via a scalable |
| | interface |
| Data | Processor for contextualizing preprocessed data with |
| Contextualizer | respect to physical activity status, environmental status, |
| 310 | data confidence, static biometrics, and the like. |
| Waveform | Processor for classifying (or categorizing) waveform type. |
| Classifier | |
| 320 | |
| Waveform | Processor for parameterizing, qualifying, quantifying, |
| Processor | normalizing, representing, and transforming |
| 330 | contextualized and/or classified physiological waveforms. |
| | The waveform processor output comprises motion and/or |
| | physiological parameters. |
| Assessment | Processor for generating assessments (i.e., physiological |
| Processor | assessments) based on information from the processed |
| 340 | waveforms. The assessment processor output comprises |
| | motion and/or physiological assessments. |

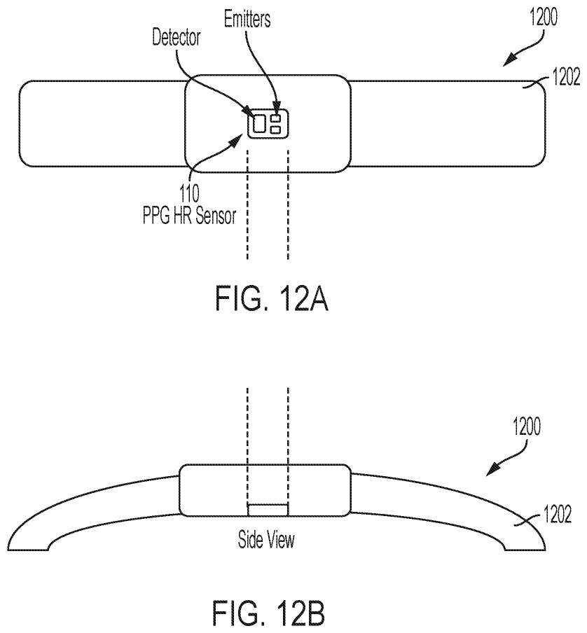

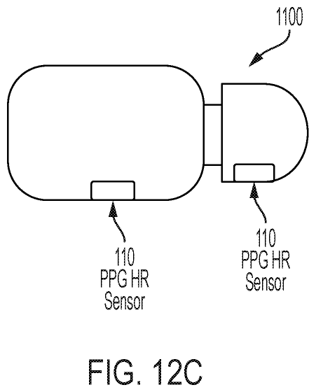

As shown in FIG. 2, the biometric waveform analysis system 10 comprises a wearable device (e.g., 1100, 1200, FIGS. 12A-12C) configured to be worn on a body of a subject, at least one metric output generator 200, at least one waveform analysis engine 300, and at least one control processor 400. The wearable device comprises a sensor system 100, wherein the sensor system 100 comprises: 1) at least one physiological sensor 110 configured to sense physiological information from the subject and generate a physiological data signal, wherein the physiological data signal comprises physiological waveform data and 2) at least one motion sensor 110 configured to obtain motion information from the subject and generate a motion data signal. The at least one metric output generator 200 is in communication with the sensor system 100 and comprises logic configured to extract at least one physiological parameter from the physiological data signal and to extract at least one motion parameter from the motion data signal.

The at least one waveform analysis engine 300 is in communication with the sensor system 100 and comprises: a) waveform capture logic configured to receive the physiological data signal from the sensor system 100 and separate the physiological data signal into a plurality of individual physiological waveforms, b) normalization logic configured to normalize the individual physiological waveforms, c) contextual logic configured to receive the motion and/or environmental data signals from the sensor system, identify subject physical activity information from the motion and/or environmental data signals, and tag the individual physiological waveforms with subject physical activity and/or environmental information, and d) physiological assessment logic configured to extract physiological information from the individual physiological waveforms and generate a physiological assessment of the subject based on the extracted physiological and contextual information.

The control processor 400 is configured to control the sensor system 100, the metric output generator 200, and the waveform analysis engine 300. As described earlier, the use of the term processor does not mean that each of these processors represent individual microcontrollers, though that possibility is certainly afforded by the invention. For example, the processors may alternatively represent pieces of code that process dedicated instructions within a localized or distributed computing system.

The sensor system 100 of the biometric waveform analysis system 10 is in communication with the metric output generator 200, the waveform analysis engine 300, and the control processor 400. In particular, the sensor system 100 provides the metric output generator 200 and waveform analysis engine 300 with sensor information (whether raw or processed); additionally, the sensor system 100 receives control commands (i.e., instructions) from the control processor 400. The sensor system 100 may comprise at least one physiological sensor 110 and at least one motion sensor 110. Nonlimiting examples of suitable physiological sensors 110 may include: a photoplethysmography (PPG) sensor, a bioimpedance sensor, an auscultatory sensor, a camera, an electrocardiogram (ECG) sensor, an electroencephalography (EEG) sensor, a ballistocardiogram sensor (BCG), an electromyography (EMG) sensor, a body temperature sensor, an electrooculography (EOG) sensor, or the like. Nonlimiting examples of suitable motion sensors 110 may include: an inertial sensor (such as an accelerometer, magnetometer, and/or gyroscope), a piezoelectric sensor, an optical sensor, a noise reference sensor, an acoustic sensor, vibration sensor, or the like. A variety of such sensors are well known to those skilled in the art. The biometric waveform analysis system 10 of FIG. 2 may additionally comprise at least one environmental sensor 110 configured to obtain environmental information in a vicinity of the subject. Nonlimiting examples of environmental sensors 110 comprise: an ambient sensor (i.e., a sensor for sensing ambient light, humidity, and/or temperature), an airborne particle sensor (i.e., a sensor for sensing particulates and/or organisms in the air), a vapor sensor (i.e., a sensor for sensing airborne vapors and/or aerosols), an acoustic sensor (for measuring environmental or ambient sounds), a radiation sensor (for sending exposure to ionizing or non-ionizing radiation), or the like.

For PPG sensing, non-limiting examples of suitable optical emitters may include LEDs (light-emitting diodes—such as OLEDs, solid state LEDs, RCLEDs, and the like), LDs (laser diodes—such as VCSELs, edge-emitting LDs, and the like), miniaturized incandescent sources, thermal (IR) emitters, microplasma sources, arrays of optical emitters, and the like. Non-limiting examples of suitable optical detectors may include photodiodes, phototransistors, cameras, photoconductors, photovoltaic sensors, miniature photomultiplier tubes, arrays of optical detectors, and the like. Other types of emitters and detectors may be used. Ideally the optical emitters selected can be sufficiently miniaturized, emit sufficient optical energy (ideally at least 10 uW) for monitoring purposes, and powered through a miniature circuit board at voltages less than 10V. Similarly, ideally the optical detectors selected can be sufficiently miniaturized, sufficiently efficient in converting photons to an electrical signal, and powered through a miniature circuit board having a power source voltage less than 10V. Though a variety of power sources may be used, a preferred power source for wearables may comprise at least one battery and/or energy harvesting element.

As show in FIG. 3, the sensor system 100 may further comprise a preprocessor 120 (preprocessing circuitry and/or logic) configured to filter sensor data and to provide physiological waveform data of interest. The preprocessor may comprise a number of processing methods and/or circuitry, including lowpass filters, bandpass filters, active noise reference filters, and the like. These filters may be analog, digital, or a combination of both. In some cases, the processing may be analog and/or digital neural network processing. Exemplary methods of active filtering and active noise reference filtering, for creating cleaner physiological waveform data by removing motion noise signals and/or environmental noise signals from the physiological signal, have been previously described by Valencell in U.S. Pat. Nos. 8,157,730; 8,652,040; 8,700,111; 8,788,002; and 8,647,270, and U.S. Patent Application Publication No. 2018/0199837; which are incorporated herein by reference in their entireties. It should be noted in that in some cases a single physical sensor 110 may comprise multiple sensors. For example, a PPG sensor may comprise multiple sensor elements (such as optical and inertial sensing elements) and be configured to sense optical scatter from blood flow, body motion, and environmental light exposure. In such case, a signal collected under one PPG sensor configuration may be processed to remove noise from a signal collected under another PPG sensor configuration. For example, ambient light exposure can be monitored with a PPG sensor having the optical emitter turned off, while at the same time, this measured and stored (i.e., in memory or via a capacitor) ambient light exposure can be used to remove the ambient light noise component of the PPG signal when the optical emitter is turned on. Such a method is described in U.S. Pat. No. 8,888,701, which is incorporated herein by reference in its entirety.

The metric output generator 200 is in communication with the sensor system 100, the waveform analysis engine 300, the relationship processor 500, and the control processor 400. In particular, the metric output generator 200 receives sensor information from the sensor system 100, communicates information between the waveform analysis engine 300, communicates information between the relationship processor 500, and receives control commands from the control processor 400. As shown in FIG. 3, the metric output generator 200 may comprise a high-rate metric processor 210 and a data formatter 220. As summarized in Table 2, the high-rate metric processor 210 may comprise circuitry and/or computing instructions for generating: a) real-time metrics, such as various physiological and motion parameters: i.e., artifact-reduced streaming PPG, heart rate, respiration rate, RR-interval, pulse wave volume, motion cadence, activity status, and the like and/or b) generating real-time time biometrics assessments (based on the real-time metrics). As a specific example, the metric output generator 200 may receive a clean, filtered PPG sensor signal (i.e., with motion noise attenuated as described earlier) and then process this clean PPG signal to generate a real-time heart rate metric (for example, using methods as presented in U.S. Pat. No. 9,314,167; U.S. Patent Application Publication No. 2017/0332974, and/or in U.S. Pat. No. 9,801,552, which are incorporated herein by reference in their entireties. Other PPG metrics may be generated in real time as well, using the aforementioned methods, as described in U.S. Patent Application Publication No. 2016/0029898, which is incorporated herein by reference in its entirety. These generated metrics and assessments are formatted by the data formatter 220 such that they can be communicated via a scalable interface, such as an application programming interface or the like. Without this formatting, it may be difficult to scale data communication between the wearable biometric waveform analysis system 10 and another processor or a remote device. For example, without the data formatter 220, multiple programming interfaces may be required for each new processor or each new remote device, making standardization of communication challenging.

It should be noted that for the biometric waveform analysis system 10 of FIG. 3, the at least one metric output generator 200 may contain logic further configured to generate a multiplexed serial data output of comprising at least one physiological parameter, at least one motion parameter, and/or at least one biometric assessment. Namely, the output of the metric output generator 200 may be multiplexed as described in U.S. Pat. No. 9,314,167 and U.S. Patent Application Publication No. 2018/0360386, which are incorporated herein by reference in their entireties.

The waveform analysis engine 300 is in communication with the sensor system 100, the metric output generator 200, the relationship processor 500, and the control processor 400. In particular, the waveform analysis engine 300 receives sensor information from the sensor system 100, communicates information with the metric output generator 200, communicates information with the relationship processor 500, and receives control commands from the control processor 400. As shown in FIG. 3, the waveform analysis engine 300 may comprise a data contextualizer 310, a waveform classifier 320, a waveform processor 330, and an assessment processor 340. As summarized in Table 2, the data contextualizer 310 comprises a processor for contextualizing preprocessed data with respect to physical activity status, environmental status, data confidence, static biometrics, and the like. The waveform classifier 320 comprises a processor for classifying (or categorizing) waveform type. The waveform processor 330 comprises a processor for parameterizing, qualifying, quantifying, normalizing, representing, and transforming contextualized and/or classified physiological waveforms. As discussed earlier, the data contextualizer 310, the waveform classifier 320, and the waveform processor 330 may operate in series or in parallel. The waveform processor 330 output comprises motion and/or physiological parameters which then feed into the assessment processor 340. Exemplary operations of the waveform processor 330 may include: parameterizing waveforms, qualification of waveforms, quantification of waveforms, normalization and/or averaging of waveforms, or creating representations and/or transforms of waveforms (such as spectral, wavelet, or other representations or transforms). The waveform processor 330 may process individual waveforms or a plurality of waveforms (such as group of consecutive waveforms or a group of selected waveforms). Additionally, it should be noted that the waveform processor 330 may processes both the AC (pulsatile) and DC (non-pulsatile) components of a signal, as opposed to just the DC or just the AC component of a signal. The assessment processor 340 comprises logic for generating assessments (i.e., physiological assessments) based on information from the processed waveforms. Exemplary assessments generated by the assessment processor may include: blood flow, cardiac output, blood pressure, blood hydration level, hematocrit concentration, blood oxygenation, the presence of arrhythmia (i.e., such as atrial fibrillation), subject infection status (such as the probability of infection in the subject by a virus, bacteria, fungus, harmful protein, toxin, or the like), blood glucose concentration, cardiac functioning (as with different chambers of the heart), cognitive status or functioning (such as cognitive load, intent, mental processing capability, awareness, and the like), psychological status (such as mood or emotion), likelihood of a cardiac event, likelihood of the onset of a stroke or aneurism, body temperature, metabolic status, the probability of a near-term spike or drop in a metabolite (such as blood glucose), inflammation status, likelihood of having cancer, cardiopulmonary functioning, or the like.

It should be noted that the waveform processor 330, as well as the other processors within the waveform analysis engine 300, may process a plurality of waveforms from a plurality of sensors in parallel. For example, the assessment of the likelihood of the onset of a stroke or aneurism may require multiple sensor locations in order to generate such an assessment. As a specific example, a PPG sensor located at multiple locations (such as with sensors integrated within 2 earpieces, with at least one PPG sensor in each ear) may be required so that the waveform processor 330 may process differential blood pressure readings between the PPG sensors, which may be indicative of blood vessel pathologies, such as clogged arteries or weakened vessels. Various methods of generating blood pressure assessments via the waveform analysis engine 300 are presented herein.

The relationship processor 500 is in communication with the waveform analysis engine 300, the metric output generator 200, and the control processor 400. In particular, the relationship processor 500 receives information from the waveform analysis engine 300 and from the metric output generator 200, and the relationship processor 500 receives control commands from the control processor 400. As described in Table 2, the relationship processor 500 is configured to generate at least one relationship between an output from the metric output generator 200 and an output from the waveform analysis engine 300. This relationship may then be presented to the metric output generator 200 (i.e., via the control processor 400) to enable the metric output generator 200 to output a real-time assessment (i.e., for an assessment that otherwise would have taken more time to generate by the waveform analysis engine 300). The relationship processor 500 may also be utilized to generate a new assessment for the subject which is initially not part of the programming of the assessment processor; this can be accomplished by the relationship processor's processing of physiological waveforms from the subject along with contextual information about those waveforms (i.e., as provided by the data contextualizer 310) over time to generate a new autonomous assessment for the subject.

The actuator(s) 600, e.g., a mechanism designed to automatically actuate upon command, are configured to actuate based on a signal from the control processor 400. Nonlimiting examples of actuators may include actuation mechanisms which are electromagnetic (i.e., optical or some other wavelength range in the electromagnetic spectrum), electrical, magnetic, mechanical, thermal, acoustical, or the like. A specific example of an actuator would be a mechanical valve in an earpiece which would actuate to seal off the ear canal (or, reversely, would actuate to open up the ear canal) in response to a signal from the control processor. Nonlimiting examples of controllable wearable actuators 600 are presented in U.S. Patent Application Publication No. 2017/0119314, which is incorporated herein by reference in its entirety.

The control processor 400 of FIG. 2 is in communication with the sensor system 100, the metric output generator 200, the waveform analysis engine 300, the relationship processor 500, and the actuator(s) 600. As summarized in Table 2, the control processor 400 is configured to communicate between and control various processors in the system 10. In particular, the control processor 400 may be adaptive and receive information from at least one of the processors and then process this information to send at least one control command to at least one other processor. Alternatively, the control processor 400 may be preprogrammed with commands to be sent to various processors. There could be a combination of adaptive and fixed processing within the control processor 400. The control processor 400 may utilize nonlinear methods, such as machine learning, to receive information and then send commands adaptively to various other processors.

Additionally shown in FIG. 2 and FIG. 3 are input/output nodes N1, N2, N3. These nodes N1, N2, N3 are illustrative only and are presented to emphasize the key inputs and/or outputs of the waveform analysis engine, from a functional perspective. These nodes may be particular useful for interfacing with other systems (via hardware or software), such as remote systems or other computational systems. Remote systems (such as smartphones, wireless hubs, remote servers, the cloud, wireless devices, the like) may be configured to send information, programs, or commands to the biometric waveform analysis system and/or configured to receive information, programs, or commands from the biometric waveform analysis system. In particular, an application programming interface (API) may be provided for each of these nodes N1, N2, N3, to facilitate scalable communication between the biometric waveform analysis system 10 and another system which can utilize the biometric data generated. As another example, the control input node N2 may communicate with a mobile application which is utilized by the subject wearing the sensor system 100. This enables the subject (or someone else) to feed manual input into the biometric waveform analysis system. It also enables an automated program to control or feed manual input into the biometric waveform analysis system 10. These nodes N1, N2, N3 should not be viewed strictly as physical connections but are rather illustrative of the functionality. For example, communicating to and from the biometric waveform analysis system 10 may comprise a single interface (such as a common API or common serial interface) representing the combination of each of these nodes N1, N2, N3. As a specific example, a single Bluetooth interface may collect information from the metric output node N1, collect information from the waveform analysis output node N3 and send information to the control input node N2.

It should be noted that a variety of analog and/or digital processing methodologies may be employed in the system 10 of FIGS. 2 and 3. Depending on the desired assessment and the quality of waveform received by the waveform analysis engine 300, the processing may be linear or nonlinear. In some cases, the processing methodologies may employ neural networks, random forests, binary trees, artificial intelligence, or other machine learning based processing. In a preferred embodiment, a combination of classical computing and machine learning would be employed where most useful. For example, generating streaming biometrics from the metric output generator 200 may be more conducive to classical processing, whereas the waveform analysis engine 300 may be more conducive to neural processing.

The biometric waveform analysis system 10 may be integrated into a variety of wearable form-factors worn at the ear, head, neck, arm, wrist, hand, digits (fingers and/or toes), leg, foot, torso, and virtually anyplace on the body with suitable coupling for the relevant sensor of interest. Various sensor elements well known to those skilled in the art can be integrated into earpieces (headsets, headphones, hearing aids, ear jewelry, etc.), wrist devices (wristbands, smartwatches, etc.), rings, bandages, patches, bands (worn at the arm, leg, etc.), body art (such as paint or tattoos) and the like. The invention may also be practiced through a remote (not necessarily wearable) sensing system, configured to analyze at least one portion of the body of the subject, such as a camera (whether optical, thermal, or other electromagnetic radiation), a laser scanner, a microphone, an acoustic or ultrasound scanner, an RF scanner, a terahertz scanner, or the like.

I. Data Contextualizer Examples

As shown in FIG. 3, the data contextualizer 310 may be configured to process data from the sensor system 100 and/or metric output generator 200 such that the extracted and/or processed physiological waveforms may be contextualized. Nonlimiting examples of contextualization processing may comprise:
Tagging activity and/or environmental profiles to waveforms
Tagging physiological cycle status to waveforms
Tagging perfusion to waveforms
Tracking confidence or signal quality to waveforms
Tagging biometric Identification to waveforms
Tagging user inputs (static biometrics & diary)
Tagging a “being worn” status to waveforms
   Contextualization may provide the waveform analysis engine 300 with key information that may help in processing the waveforms and in generating physiological assessments from the waveforms. It should be noted that data contextualization for a waveform requires that the waveform be extracted (i.e., identified) from signals generated by the sensor system 100. The step of extracting may be implemented by the metric output generator 200 (such as with the high-rate metric processor 210) or the waveform analysis engine 300 (such as with the data contextualizer 310 itself). Understandably, extracted waveforms and contextual information (collected by sensors in the sensor system) must be sufficiently time-correlated, such that the extracted waveform and the associated contextual information may be sufficiently physiologically related to each other.

Specific examples of waveform contextualization may be outlined by considering the case where the sensor system 100 comprises a PPG sensor 110 and a motion sensor 110, wherein PPG waveforms are extracted (as described above) and contextualized for further processing. In some nonlimiting cases, the extracted waveforms may be processed differently, depending on the context provided for those waveforms, such that the accuracy of an assessment (i.e., as processed by the assessment processor) is improved. In other nonlimiting cases, the context provided for waveforms may be used to generate an assessment that cannot be generated at all without the provided context. For example, environmental information in the vicinity of a subject (i.e., as sensed by an environmental sensor) may be processed by the data contextualizer 310 to tag (i.e. label) a PPG waveform with environmental context. As a specific example, data from an ambient temperature sensor (from the sensor system 100) may be used to provide ambient temperature context to an extracted PPG waveform, such that these waveforms may be processed in context of an ambient temperature reading. In such case the PPG waveforms tagged as being “high ambient temperature” waveforms may be processed differently by the waveform processor than “low ambient temperature” waveforms. This tagging may be particularly useful because the ambient temperature around a person may affect the shape of the PPG waveforms of that person and hence may affect the assessment generated for that person via the assessment processor 340. Therefore, tagging these waveforms with ambient temperature readings may be critical for a sufficiently accurate physiological assessment, such a PPG-based assessment of blood pressure or cardiac output. Alternatively (or additionally), the ambient temperature sensor readings in this specific example may be used to provide context for a physiological assessment where the waveforms are not necessarily processed differently but rather the overall assessment generated relies on the ambient temperature information. For example, an assessment of the onset of heat exhaustion may require processing PPG waveforms to generate blood flow and blood pressure information and may also require ambient temperature information for context that heat exhaustion is indeed probable.

Activity profiles are another example of contextualization that may be applied to waveforms. For example, generating an assessment of blood pressure from a PPG waveform may require activity context for improving the accuracy of the blood pressure assessment. This is because the PPG waveform is chiefly indicative of blood flow information (as opposed to blood pressure information), and so the transfer function between the blood flow information and blood pressure information may be nonlinear. Thus, processing PPG waveforms to generate a blood pressure assessment at rest (very low physical activity) may not be the same as processing PPG waveforms to generate a blood pressure assessment during higher physical activity. In such case, the data contextualizer 310 may be configured to tag activity profiles to the extracted PPG waveforms. The waveform processor 330 may then parameterize the PPG waveforms for a blood pressure assessment in the assessment processor, but the blood pressure assessment may require knowledge of physical activity in order to generate an accurate assessment of blood pressure, as the relationship (i.e., transfer function) between PPG waveform parameters and blood pressure may be physical activity dependent. As a specific example, the waveform analysis engine 300 may employ a machine learning model configured to generate a blood pressure assessment from the activity-contextualized PPG waveforms, and the machine learning algorithm used to generate a blood pressure assessment from the PPG waveform may thus be dependent on the physical activity status of the subject.

The system 10 affords numerous ways of leveraging contextualized physiological waveforms to generate or to improve assessments for the subject. For example, it may be useful to tag the physiological cycle status (i.e., the circadian rhythm status) to physiological waveforms to generate or improve an assessment for the subject. Knowing that a subject is at their peak circadian cycle (highest daily metabolism) may be important for an assessment of how well someone is responding to a therapy (i.e., a medication). There are at least 2 reasons for this: 1) it may be important to assess sensor readings at multiple times of the day in context of a subject's circadian status and 2) the physiological waveforms may need to be processed differently depending on the circadian status of the subject. As a specific example of #1, an assessment of weekly blood pressure trending in response to a blood pressure medication may require that a subject's blood pressure readings (as generated by from PPG waveforms) be averaged for multiple times of the day or for multiple points in the circadian cycle (multiple circadian statuses) of a subject. As a specific example of #2, an assessment of mental health trending in response to a psychological or psychiatric therapy (such as a behavioral health plan or medication) may require that the EEG, PPG and/or ECG waveforms of the subject be processed differently depending on the time of day or circadian status of the subject—for example, in making the assessment, more or less weight may be given for points in the circadian cycle associated with the highest or lowest metabolism of the subject.

Another example of leveraging contextualized physiological waveforms to generate or improve an assessment for the subject is for generating an assessment of hypovolemia or blood hydration for the subject. In such case, the metric output generator 200 may provide a real-time perfusion assessment to the waveform analysis engine 300. As a specific example, the blood perfusion may be generated by the metric output generator 200 processing the PPG spectral amplitude at the subject's heart rate (i.e., pulse frequency), wherein the real-time blood perfusion is directly proportional to the PPG spectral amplitude multiplied by the real-time heart rate. The data contextualizer 310 may then provide perfusion context to the extracted PPG waveforms. In such case, the waveform processor 330 may parameterize or qualify PPG waveforms differently depending on the perfusion context. For example, waveforms having low perfusion may be nosier and hence require the waveform processor 330 to parameterize the noisy signals using waveform parameters that are less susceptible to noise (such as integrals of a waveform as opposed to derivatives of a waveform). Thus, an assessment of hypovolemia or low blood hydration may be more accurate leveraging perfusion context.

As a further example of leveraging the contextualized physiological waveforms to generate or improve an assessment for the subject, the data contextualizer 310 may be used to provide context that a subject is in a low state of physical activity and breathing periodically (i.e., not erratically). The waveform classifier 320 may then be applied to isolate physiological waveform features associated with inhalation vs. waveform features associated with exhalation during periodic breathing (respiring). This information can be especially useful for machine learning algorithms that are executed via the assessment processor 340. As a specific example, the assessment processor 340 may be executing a machine learning algorithm that is configured to receive input from a subject PPG sensor and a subject motion sensor to output an estimation for subject blood pressure (such as systolic, diastolic, or mean arterial blood pressure), without requiring a calibration (such as a calibration via a blood pressure cuff) by the user. In such case, the machine learning algorithm must guess the blood pressure for the subject without “ground truth” provided by the user, and thus heuristic features (features spawned from known relationships between the PPG waveform and BP) may be critical for sufficient estimation accuracy. Because it is well known that inhaling decreases blood pressure and increases heart rate, and that exhaling increases blood pressure and decreases heart rate, the contextualized and classified waveforms can be applied to the machine learning algorithm to update coefficients based on the known respiration information. Namely, although an ideal machine learning algorithm should predict an increase/decrease in blood pressure during exhalation/inhalation respectively, this may not always be the case without ground truth BP cuff information regularly updated. Fortunately, if inhalation and exhalation are properly classified in the PPG waveforms, the machine learning algorithm can be more intelligent and adapt to update the algorithm coefficients—based on the a priori knowledge that BP estimations should go up/down with exhalation/inhalation. Thus, when initial model coefficients do not yield the proper trending, the coefficients may be corrected intelligently, such that BP estimation accuracy may literally improve with each breath. Phrased another way, with appropriately contextualized and classified PPG waveforms, the machine learning algorithm can adapt with breathing information to become more accurate without a calibration, using a priori knowledge about the relationship between breathing and blood pressure. (In principle, one may assume a normal change in blood pressure with breathing between 5 mm and 10 mmHg.) Knowing that the subject is at rest and in a state of periodic breathing may be critical for this application, as experimentation has shown that machine learning models may not be able to make sense of PPG waveforms corrupted by motion artifacts or erratic breathing artifacts.

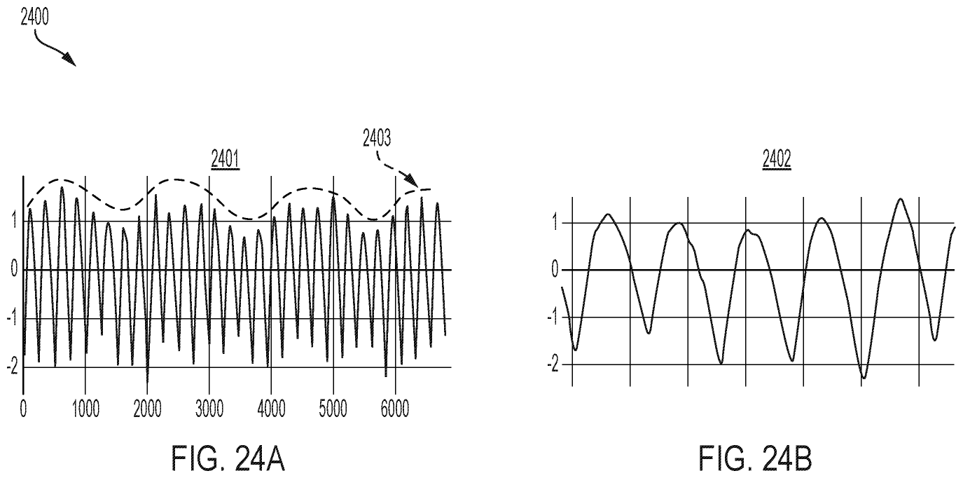

Numerous methods may be used to classify inhalation vs. exhalation for properly contextualized waveforms including those described by Addison et al. in “Developing an algorithm for pulse oximetry derived respiratory rate (RRₒₓᵢ): a healthy volunteer study”, which is incorporated herein by reference in its entirety. (https://www.researchgate.net/publication/221734860_Developing_an_algorithm_for pulse_oximetry_derived_respiratory_rate_RRoxi_A_healthy_volunteer_study). Considering an exemplary set of PPG waveforms collected during periodic breathing, such as PPG waveforms 2401 and 2402 of FIGS. 24A and 24B, a pulse picker, zero-crossing methodology, envelope detector, or other pulse identification algorithm or circuit may be applied to classify the respiration envelope (such as envelope 2403, FIG. 24A) overriding the heart beat peaks. For the particular example of FIG. 24A, the rising edge of the envelope represents exhalation, and the falling edge represents inhalation.

Another key value of contextualizing physiological waveforms is that the waveforms may be individually tagged with waveform confidence information (i.e., confidence that the extracted waveforms can be trusted as being accurate), and this confidence tagging may be used throughout all future processing of the tagged waveforms. Waveforms tagged with low confidence may be processed differently than waveforms tagged with high confidence. In some assessments, waveforms tagged with low confidence may be completely ignored in generating the assessment, such as not to taint the assessment for the subject. In other cases, lower confidence waveforms may be essential, a necessary evil, such that they cannot be simply ignored as long as they are of sufficient confidence, as in the case where waveform quality is extremely poor (perhaps due to motion artifacts from high physical activity or due to poor sensor coupling) and waveforms of sufficiently quality are scarce. In such case, any sufficient waveform, even if below the most desired confidence, may be welcome for the assessment. Thus, waveforms tagged with low confidence may be processed differently by the waveform analysis engine 300, using higher-power-consuming algorithms, such as advanced machine learning algorithms or advanced digital filters, to generate an accurate assessment for the subject. Altogether, this enables the biometric waveform analysis system 10 to utilize smart power savings for using higher power in processing lower confidence waveforms and using lower power in processing higher confidence waveforms. It should be noted that this example is not enabled by simply providing a confidence reading, such as a sensor integrity reading, for processed metrics—as with providing heart rate values along with a sensor integrity value to a mobile application. In contrast, this example is aimed at tagging confidence in the physiological waveform itself or a group of waveforms as opposed to a processed metric. Indeed, a high-integrity series of heart rate measurements can be generated with either low- or high-quality PPG or ECG waveforms; thus, confidence in a metric is not identically equal to confidence in a waveform.

Data contextualization may also leverage information that may be input by the subject or another person to provide context that will improve the accuracy of an assessment or enable the assessment altogether. For example, static biometric information—such as weight, height, age, gender, and the like—may be tagged to waveforms by the data contextualizer, and this information can help improve assessments made for that user. As a specific example, machine learning assessments of cardiac output and blood pressure can be improved by providing static biometric information to the algorithms running in the assessment processor 340. Other information about the user may comprise manual inputs from the subject (or from others). For example, through a mobile device, in communication with the biometric waveform analysis system 10, the subject may input daily information about what they are doing, what they are eating or drinking (i.e., food diary information), and when they are feeling stressed, thirsty, hungry, tired, in pain, and the like. This data may be tagged as contextual data to the respective physiological waveforms to provide assessments for the subject which combine processed waveform information as well as subject input information. As a specific example of this, digestive auscultatory waveforms sensed by an auscultatory sensor may be processed with subject input about hunger, and a relationship between auscultatory waveforms indicative of digestive sounds and subject input of hunger may be used to generate an assessment of a digestive disorder or other medical condition.

In some cases, the data contextualizer 310 may contextualize waveforms for the purpose of generating a new assessment for the user over a period of time, thereby generating a new relationship (i.e., via the relationship processor 500) that is not current to the assessment processor 340. Over time, contextual input from sensors 110, subject manual inputs, and/or automated inputs from an external device may be tagged with physiological waveforms such that new relationships between the contextual information and waveform information may be developed. For example, contextual user inputs rating the intensity of pain over a period of time may be processed with physiological waveform data over that same period of time to identify a relationship between the physiological waveform data and pain intensity. This relationship may then be programmed into the assessment processor 340 to generate an autonomous assessment of pain intensity for the subject.

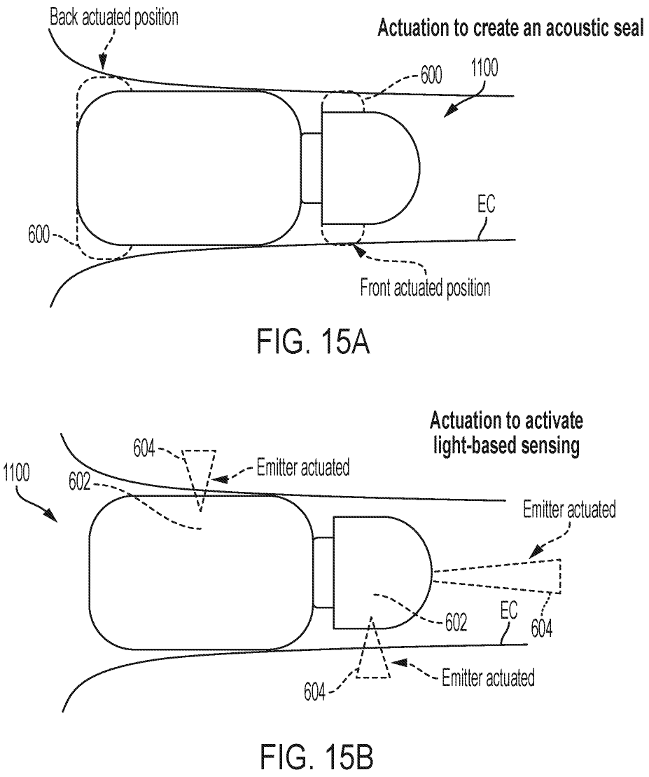

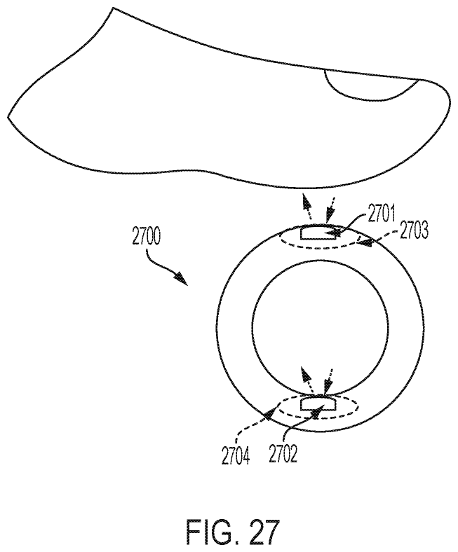

In some cases, the data contextualizer 310 may contextualize waveforms based on sensor location, ultimately to improve the accuracy of a biometric assessment (e.g., as determined by the assessment processor 340). This may be particularly useful for PPG sensors, imaging sensors, and auscultatory sensors, as (unlike the case for ECG, EEG, and other electrode-based sensors) useful information from these types of sensors may be picked up from a single sensor at a single location of the body. A specific example is illustrated through a digit-worn apparatus 2700 (e.g., a ring) shown in FIG. 27. The ring 2700 comprises: 1) a PPG sensor 2701 configured to sense physiological data from a finger (or other body part) placed upon the top of the ring, 2) a PPG sensor 2702 configured to sense physiological data from the finger on which the ring is being worn, 3) an electrode 2703 surrounding the PPG sensor 2701 configured to sense electrical information from a finger (or other body part) placed on the top of the ring, and 4) an electrode 2704 surrounding the PPG sensor 2702 configured to sense electrical information from the finger on which the ring is being worn. In one implementation of the ring device 2700, the accuracy of a blood pressure estimation may be increased by processing contextualized PPG data from both the PPG sensors 2701 and 2702, as opposed to just one of the sensors. This may be achieved by the data contextualizer 310 tagging sensor data based on sensor location, such that the assessment processor 340 can process PPG information from each sensor location in context of sensor placement. The inventors have discovered that orthogonal information exists in PPG signals from one finger on one hand vs. another finger on the opposing hand. (Additionally, this trend holds for any 2 or more PPG sensors worn on 2 or more body locations that are sufficiently apart from each other.) This is because the pressure information from PPG is dispersed along the body in proportion to distance from the aorta of the subject, such that no two body locations solely comprise the same identical pressure information. Thus, the assessment processor 340 may implement a machine learning algorithm that is capable of accepting sensor location information as means to contextualize the PPG data and thus provide orthogonal information in estimating subject BP. It should be noted that this improvement in accuracy has been demonstrated by the applicant without implementing time-alignment, as is required with pulse-transit-time analysis (PTT analysis). In contrast, the PPG waveform features alone, which are somewhat different between each body location, have been found to be sufficient to provide discrimination between blood pressure estimations. It should also be noted that the electrodes 2703 and 2704 may be used for ECG, EEG, EOG, EMG, EEG, bioimpedance, and the like, such that (depending on the body location) these measurements may be collected in parallel with PPG measurements. Lastly, it will be appreciated that this innovation may also be practiced via the devices of FIGS. 15A-15B, 16A-16C, and 17A-17B, as well.

In some cases, the data contextualizer 310 may contextualize waveforms for the purpose of biometric identification of those waveforms. In such case, the waveforms may be tagged with biometric identification information about the subject (i.e., manual input from the subject regarding the subject's identification) such that assessments for the subject may be more personalized to the subject. Interestingly, when combined with the relationship processor 500, this contextual information may be used to generate a model that can identify the subject without manual input using sensor data alone. As a specific example, the biometric waveforms and inertial waveforms from a subject may be related in a characteristic way, as when the intensity of a PPG signal collected from a wrist-PPG sensor changes substantially when a subject moves their wrist up or down. This historical contextual information may feed the relationship processor 500 to generate a real-time biometric identification for the user wearing the wrist-PPG sensor.

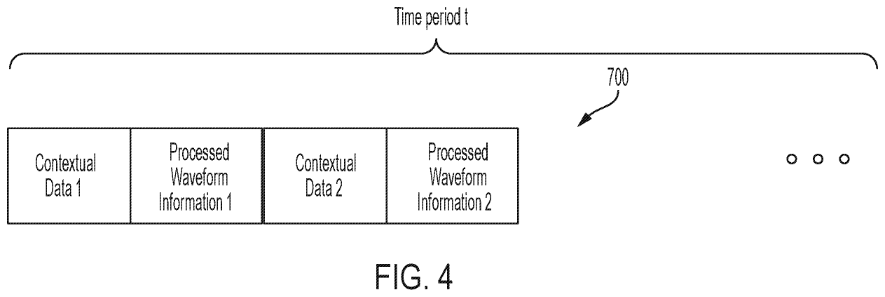

Data from the data contextualizer 310 may be serialized into a serial data stream 700 as shown in FIG. 4. In such case, the contextual data information may be tied to one or more sets of waveforms.

II. Waveform Classifier Examples

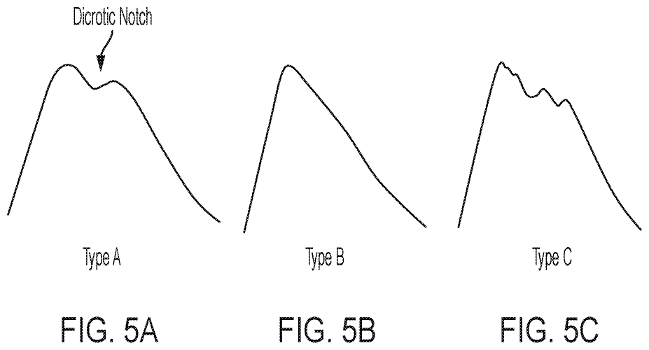

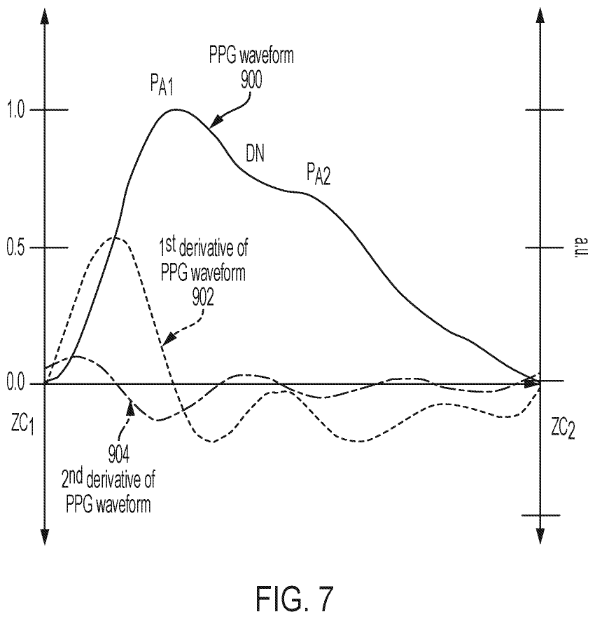

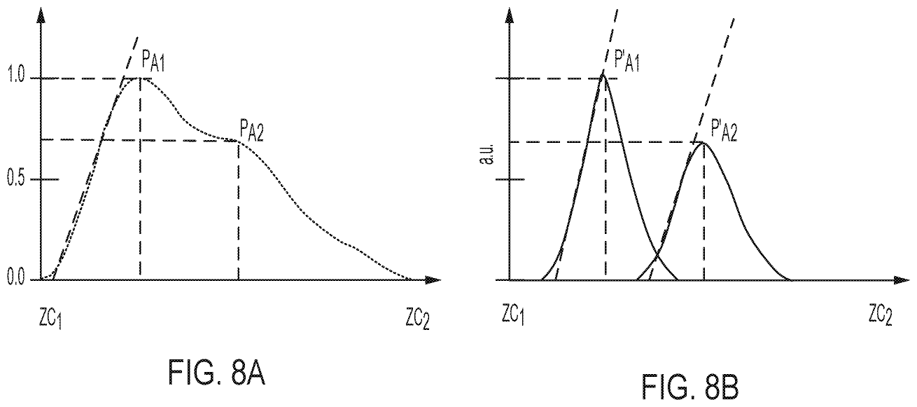

Physiological waveforms may comprise distinct features which may fall into a class (a category or type). For example, physiological waveforms may be categorized as normal or abnormal. And within these categories, normal and abnormal waveforms may comprise numerous subcategories. As specific example, FIGS. 5A-5C show examples of three classifications of PPG waveforms. Type A (FIG. 5A) and Type B (FIG. 5B) waveforms comprise normal PPG waveforms. (Note: These waveforms have been normalized and stripped of the non-pulsatile (DC) signal such that they can be more easily compared.) In type A, the dicrotic notch is much more pronounced than that in Type B, for which the dicrotic notch is barely perceptible at all. In contrast, the Type C (FIG. 5C) waveform comprises an abnormal PPG waveform, in this case a PPG waveform exhibiting pulsus bisferiens (also known as biphasic pulse), indicating an aortic waveform with 2 peaks per cardiac cycle. This abnormality may indicate an issue with the aortic valve, such as regurgitation or stenosis, or a cardiac muscle issue (cardiomyopathy). The waveform classifier 320 may employ a method of processing photoplethysmography (PPG) waveforms generated by a PPG sensor 110 worn on a body of a subject, the method comprising: obtaining a PPG waveform from a PPG data signal from the PPG sensor, classifying the PPG waveform according to PPG waveform type, and processing the PPG waveform using a processing method selected based on the waveform type of the PPG waveform. Thus, the waveform classifier 320 may be configured to classify the individual physiological PPG waveforms as normal or abnormal. Moreover, the waveform classifier may be configured to classify an abnormal PPG waveform as having bisferiens. This generated classification for each waveform may be stored and tagged along with the associated waveform, such that processing assessments can be streamlined. For example, in generating an assessment of blood pressure, the assessment processor 340 may process a bisferiens waveform differently than a normal waveform, as the relationship between the subject PPG signal, such as the normalized waveform peak locations shown in FIG. 7 and FIG. 8, and subject blood pressure may be different depending on these two classifications.

It should be noted that the aforementioned example is exemplary only and not intended to be limiting to other forms of classification. Namely, other waveform types may be classified by the waveform classifier via classifier logic. For example, the logic of the waveform classifier 320 may be configured to classify the individual physiological waveforms as one of a plurality of normal waveforms, such as the normal waveforms of Type A and Type B, as shown in FIGS. 5A and 5B, or the like. Moreover, the waveform classifier 320 and the method described above may be employed to classify numerous types of waveforms, not limited to PPG waveforms, such as normal and pathological waveforms from ECG or EEG.

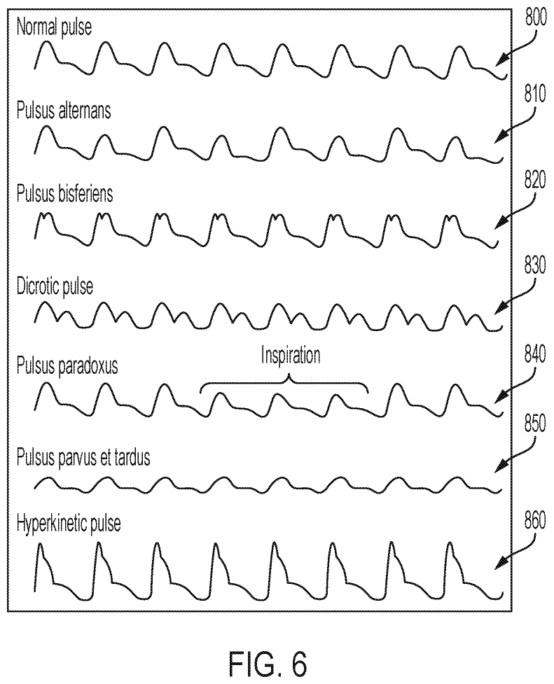

In some cases, information about a single waveform itself may only be made manifest by analyzing a series of consecutive waveforms. As a particular example, FIG. 6 shows a summary of known PPG waveform patterns 800-860 (taken from https://www.sciencedirect.com/topics/medicine-and-dentistry/pulsus-alternans). Many of the patterns (pulsus bisferiens 820, dicrotic pulse 830, pulsus parvus et tardus 850, and hyperkinetic pulse 860) presented in FIG. 6 may be identified by processing a single PPG waveform. However, normal pulse 800, pulsus paradoxus 840 and pulsus alternans 810 may require processing several consecutive pulses in order to generate an assessment that one of the many consecutives pulses is classified (tagged) as “normal”, having “pulsus paradoxus”, or “pulsus alternans”. Thus, the waveform classifier 320 may be configured to buffer and process a batch of a plurality of consecutive waveforms, determine a waveform pattern or key PPG features, and then classify at least one of the waveforms accordingly (i.e., by waveform type or by key PPG feature). This classification may then be tagged to at least one of the associated waveforms stored in memory. As another specific example, the waveform classifier 320 may ascribe arrhythmia (or perhaps more specifically atrial fib) to a serious of consecutive PPG and/or ECG waveforms, and then tag the classification of arrhythmia to at least one of the associated stored waveforms.

As another specific example, the waveform classifier 320 may process a batch of buffered (stored) PPG waveforms, determine that the waveforms show a “normal” pattern, and then classify at least one of the waveforms as “normal” (i.e., as belonging to at least one of a plurality of normal waveforms types). In these specific examples, the final assessment of “pulsus paradoxus”, arrhythmia, normal condition, etc. may be generated by processing the classified waveform information along with contextual information and other information collected by the sensor system 100 and processed by the waveform analysis engine 300. For example, an exceptionally increasing PPG waveform magnitude during exhaling, followed by an exceptionally decreasing PPG waveform magnitude during inhaling, may be classified as expressing pulsus paradoxus by the waveform classifier 320. However, via activity characterization, the data contextualizer 310 may indicate that the subject is undergoing a controlled breathing stress reduction exercise, or that the subject is in a state of regular breathing, and this combined context and waveform class information may be used to make an assessment that the subject is not experiencing pulsus paradoxus but rather is in a state of meditation or high relaxation.

III. Waveform Processor Examples

As shown in FIG. 3, the waveform processor 330 may be configured to process waveform data from the sensor system 100 and/or metric output generator 200. Examples of such waveform processing may comprise:
Parameterization of waveforms
Qualification of waveforms
Quantification of waveforms
Normalization or averaging of waveforms
Representation of waveforms
Transforms of waveforms
   The waveform processor 330 may work sequentially to the data contextualizer 310 and waveform classifier 320, and it may also work in parallel, depending on the specific processing methodology.

A specific example of normalization, parameterization, and transforms of waveforms via the waveform processor 330 is presented in FIG. 7. FIG. 7 illustrates a processed PPG waveform 900 which has been normalized in amplitude in the y-axis (with a peak amplitude set to “1”) and normalized in time in the x-axis (with a start point and stop point of ZC1 (zero-crossing #1) and ZC2 (zero-crossing #2) respectively). The parameters PA1 and PA2 represent the calculated systolic and diastolic peaks respectively. The region in between these peaks, DN, is the diastolic notch parameter that may also comprise blood pressure information about the subject. Additionally, the normalized PPG waveform 900 may be processed into Gaussian counterparts by fitting the normalized PPG waveform 900 to Gaussian curves, as shown in FIG. 8B. In such case, the position of the peaks P′A1 and P′A2 in the normalized timescale may be useful parameters for generating physiological assessments. Two transforms are presented in FIG. 7, including the 1^{st} derivative 902 of the PPG waveform 900 and the 2^{nd} derivative 904 of the PPG waveform 900. Each of these transforms 902, 904 may be useful in assessing hemodynamics for the subject. As with the Gaussian-fit peaks (P′A1 and P′A2) of the normalized PPG waveform 900 illustrated in FIG. 8B, Gaussian curves may also be fit to these derivative transforms, and the position of the resulting peaks on the respective normalized timescales may be useful in assessing hemodynamics for the subject.

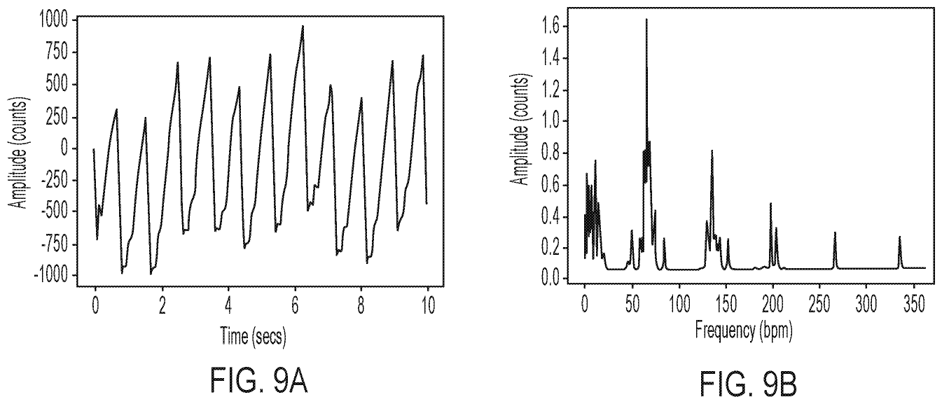

A specific example of a transform that may be generated by the waveform processor is presented in FIGS. 9A-9B. FIG. 9A shows 11 PPG waveforms taken from a subject wearing a PPG sensor and FIG. 9B shows the spectral transform of the 11 waveforms, with a speak spectral amplitude at the heart rate of the subject (˜70 BPM). The lower frequencies (below 20 BPM) are primarily indicative of the subject's respiration rate (breathing rate). This spectral transform information can be used by the assessment processor 340 to generate assessments for the subject. For example, the decay in the spectral amplitudes of the heart rate harmonics (all at multiples of 70 BPM) may be correlated with the subject's blood pressure and thus be used to assess the subject's blood pressure. A plurality of spectral transform features (i.e., such as the spectral amplitudes of various key frequencies) may be input into a machine learning model to correlate PPG signals with subject blood pressure.

The specific example of FIG. 7 is not meant to be limiting to these particular normalizations, parameterizations, transforms, etc. Additionally, other physiological waveforms may be parameterized in a similar fashion in this invention. Moreover, the waveform processor 330 may employ a variety of additional transforms, such as wavelet transforms, the Teager-Kaiser energy operator, chirplet transforms, noiselet transforms, spaceograms, derivatives, integrals, and the like. Additionally, the waveform analysis engine 300 may execute the waveform processing via a convolutional neural net (or other suitably deep machine learning model), wherein the convolutional neural net comprises one or more of the key processors in the waveform analysis engine 10 summarized in FIG. 3: the data contextualizer 310, waveform classifier 320, waveform processor 330, and assessment processor 340.

IV. Examples of the Biometric Waveform Analysis System in Action

### a) Processing Categorized Representative Waveforms

The biometrics waveform analysis system 10 of FIG. 2 and FIG. 3 enables a method of generating a physiological assessment of a subject based on processing representative, categorized PPG waveforms. The method comprises: 1) obtaining a photoplethysmography (PPG) data signal from a PPG sensor 110 worn on a body of the subject, 2) separating the PPG data signal into a plurality of individual PPG waveforms, 3) buffering the PPG waveforms, 4) classifying the buffered PPG waveforms into respective categories according to a physiological parameter associated with the subject and/or a temporal parameter associated with the subject, 5) processing at least two PPG waveforms within each category to generate a respective representative PPG waveform in each category, and 6) processing the representative PPG waveform in at least two of the categories to generate a physiological assessment for the subject. This method enables numerous physiological trends to be assessed for a subject by tracking representative PPG waveforms in context of a physiological or temporal parameter associated with the subject.

As an example, the buffered PPG waveforms may be classified into respective categories according to a subject biometric parameter, such as heart rate or the like, such that the PPG waveforms are classified by certain heart rate ranges (categories) associated with the PPG waveforms. The PPG waveforms in each respective category may be processed to generate a representative PPG waveform for each heart rate category. The processing may comprise numerous methods, such as averaging the waveforms, generating a transform for the waveforms (such as a spectral transform, wavelet transform, Teager-Kaiser operator, or the like, as listed earlier), integrating the waveforms, or the like. Moreover, the individual waveforms within a category may be normalized with respect to their DC PPG background (or other normalization reference) before generating the representative waveform. Regardless of the signal processing details, the ultimate goal is to generate a representative waveform for each category of the biometric parameter (in this particular example, heart rate, but the invention holds more broadly) that may then be used to generate an assessment for the subject, such as (but not limited to) an assessment of subject blood pressure, subject stress, subject infection (as with an illness associated with a virus, bacteria, harmful protein, fungus, or the like), a cardiac assessment (such as an assessment of heart health or heart disease), a pulmonary assessment (such as an assessment of pulmonary health or pulmonary disease), subject body temperature (such as core body temperature, skin temperature, or other body temperature), a cardiopulmonary assessment (such as an assessment of cardiopulmonary health or cardiopulmonary disease) and/or a circadian assessment (such as an assessment of circadian entrainment or an assessment of biometric patterns associated with a time of day).

As a specific example of the utility of the aforementioned method, a representative PPG waveform for each heart rate category may be generated for a subject over a period of time (several seconds, days, weeks, etc.), such that each category represents a heart rate range associated with the representative waveform. For example, one category may comprise a representative PPG waveform associated with lower (resting, sleeping, or meditating) heart rates, another category comprises a representative PPG waveform associated with elevated heart rates (such as during exercise or other physically active states), and perhaps another category comprises a representative PPG waveform associated with mid-range heart rate levels. Because the transfer function between the PPG signal and the blood pressure estimate for the subject may be heart-rate dependent, and because the representative waveforms are categorized, the assessment processor 340 can process the categorized waveforms with specialized algorithms tailored to the respective transfer function for that category (i.e., the transfer function most suitable for the range of heart rate values associated with the representative waveform), to generate an accurate blood pressure value for each category. Without such categorization, some PPG waveforms may be processed with inferior algorithms for that particular waveform category that are better suited for other categories of PPG waveforms, yielding less than optimal accuracy.

As another specific example of the utility of the aforementioned method, a representative PPG waveform for each biometric category may be generated for a subject over a period of time (several seconds, days, weeks, etc.), such that each category represents a circadian category (i.e., a point in time in the subject's circadian rhythm or a time-of-day). A representative waveform for each circadian category may be generated for the subject as a baseline, generated by collecting PPG information over a first period of time, such as a few days, a week, or the like. With the baseline categorized representative waveforms generated, updated categorized representative waveforms may be generated on an ongoing basis by processing PPG waveforms collected over a second period of time. The assessment processor 340 may then process the updated representative waveforms in context of the baseline representative waveforms to determine deviations which may be indicative of disease. In this example, the deviations in respective circadian categories may be the most telling about the physiological status about the person. For example, a deviation in the updated representative waveforms from the baseline representative waveform in the peak circadian category (typically around mid-day) may be indicative of a metabolic disorder. Similarly, a deviation in the updated representative waveforms from the baseline representative waveform in the lowest circadian categories (such as during sleep) may be indicative of a neurological disorder. Moreover, deviations in the updated representative waveforms from the baseline representative waveforms within one or more categories can be used to track the impact of lifestyle changes on health. For example, for a subject utilizing a digital food diary while wearing a PPG sensor 110, the subject may start a baseline PPG data collection process during a first period of time—to create baseline representative waveforms in various categories—before the start of a new diet and then update the representative PPG waveforms over a second period of time at the start of the new diet. Deviations in the baseline and updated representative waveforms in correlation with food diary may then be processed by the assessment processor 340 to provide a health assessment impact of the new diet for the subject.

As another example of the utility of the aforementioned method, the representative PPG waveforms for a plurality of known biometric categories may be generated for a subject sequentially over multiple periods of time, creating a historical record of representative PPG waveforms for the plurality of known biometric categories. The assessment processor 340 may then process the historical record to identify patterns in the data indicative of known biometric categories such that the subject's biometric category can be predicted in the future. For example, a subject history may comprise a representative PPG wave for a series of consecutive days where the known biometric categories of stable health, improving health, or declining health were recorded by a benchmark device or manually, e.g., via user input. Processing this historical data, the assessment processor 340 (or relationship processor 500) may identify a pattern connecting the representative PPG waves with the most probable biometric category and then update processing for future assessments based on this pattern, such that future PPG waveforms (for which the biometric category is unknown) may be processed in real time using this updated processing to predict the biometric category of stable, improving, or declining health.

The method above gives examples of biometric categories of “stable, improving, or declining health”, but the invention is not limited to these categories. As a specific example, the categories may be normal glucose levels, high glucose levels, and low glucose levels. In such case, the subject may be wearing a CGM (continuous glucose monitor) as the wearable sensor 110 in the sensor system 100 or be measuring blood glucose on a regular basis via a standard finger-pricking device. As with the example above, a subject history may comprise a representative PPG wave for a series of consecutive days where the known biometric categories of normal, high, and low glucose levels are recorded (either automatically or manually) for input into the biometric waveform analysis engine 300. Processing this historical data, the assessment processor 340 (or relationship processor 500) may identify a pattern connecting the representative PPG waves with the most probable biometric category, update processing based on this pattern, and then process future PPG waveforms (where the biometric category is unknown) using this updated processing. Thus, in the future, the subject may not need to wear the CGM or prick their finger in order to get a routine assessment of normal, high, or low glucose levels. Besides static estimates of current glucose category, the system 10 may also enable dynamic estimates of glucose category. For example, additional known biometric categories may comprise “glucose about to spike” or “glucose about to drop”. As long as the CGM or finger pricking method has been able to record spikes and drops in glucose during continuous PPG monitoring, a pattern connecting the representative PPG waveforms with the most probable biometric category may be generated such that the biometric waveform analysis engine 300 may be able to predict glucose spikes and drops for a subject without the subject needing to wear the CGM or prick their finger. It should be noted that, for this particular example, a multi-wavelength PPG monitor may be particularly important, as the relationship between the PPG signal and the glucose levels for a subject may be particularly complex. As one example, changes in optical absorption of the skin, due in part due to changes in blood perfusion, respiration, and/or blood oxygenation, may be associated with a future spike or future drop in in glucose, and using multi-wavelength PPG sensing can enable a determination of this change in optical absorption by detecting a change in the relative intensities of the constituent optical wavelengths in the skin-scattered PPG signal for the subject.

Another innovative application of this historical PPG record is that it may be used to generate further information about the relationship between PPG data biometrics. For example, representative waveforms may be generated for a plurality of biometric categories for a subject, such as heart rate ranges, breathing rate ranges, blood glucose ranges, subject body temperature (such as core body temperature, skin temperature, or other body temperature), blood pressure ranges, and the like. The assessment processor 340 and/or relationship processor 500 may then process these representative waveforms to generate a relationship between the PPG waveform properties and each biometric category. Thus, in future data collection, the determination of the values of each biometric category for the subject may comprise simply processing the PPG waveforms using learned relationships rather than using a plurality of high-power processing techniques to generate these values.

A key functionality of this innovation is that the wearable sensor platform comprising the biometric waveform analysis system 10 can learn from the subject autonomously, as the subject lives their daily life, and develop targeted health monitoring for the subject. Thus, health monitoring that may have required continuous heart rate monitoring, breathing rate monitoring, blood pressure monitoring, and/or some other continuous, high-frequency DSP calculation may be enabled by a non-continuous, low-frequency capture and processing of PPG waves instead. This is because a machine learning model connecting the captured streaming PPG data with the predicted streaming biometrics may be quite low-power-intensive for the DSP. This ultimately may extend the battery life of the wearable sensor solution.

### b) Processing Characteristic Features

The biometric waveform analysis system 10 of FIG. 2 and FIG. 3 may be configured to generate a characteristic feature of a physiological waveform. In turn, this may enable streamlined assessment processing by the assessment processor.

As a specific example, the waveform processor 330 may generate a parameter for sampled PPG waveforms which comprises a dicrotic notch feature, as shown in FIG. 7 and FIGS. 8A-8B. This feature may be generated through numerous different types of methods: one example may be by sampling the normalized PPG information between the two peaks PA1 and PA2 (FIG. 8A), or P′A1 and P′A2 (FIG. 8B); another example may be by arbitrarily declaring two consistent points in time (along the normalized time scale of the x-axis of FIG. 7 and FIGS. 8A-8B) as being in the dicrotic notch range and then sampling the normalized PPG information in between these two consistent points in time (arbitrarily, regardless of where the dicrotic notch may actually be for each wave). Other methods of generating the dicrotic notch feature may be used. Because the blood pressure of the subject may be related to the dicrotic notch feature, the assessment processor 340 may then process this characteristic feature to generate an assessment of blood pressure.

A key benefit of generating characteristic feature via the waveform processor 330 is that the assessment processor 340 may not need to process the entire PPG waveform to generate the assessment but rather a characteristic feature of that waveform, reducing processing resources (saving battery life) and improving the scalability of the assessment to various types of PPG sensors. Additionally, by focusing the assessment processing on characteristic features (as opposed to the entire PPG waveform), the impact of noise (such as motion noise, high-frequency noise, quantization noise, or the like) by noisier PPG features may be reduced in generating the desired assessment. Namely, the inherent noise in the characteristic features may be less than that of other features in the PPG waveform.

In some embodiments, a plurality of characteristic features may be generated by the waveform processor 330, and the plurality of characteristic features may then be used by the assessment processor 340 to generate a desired physiological assessment. As a specific example, for a biometric waveform analysis system 10 having a multi-wavelength PPG sensor 110, configured to emit and detect light at a plurality of distinct wavelengths, the waveform capture logic may be configured to receive physiological data signals from the PPG sensor 110 and separate the PPG data signals into a plurality of individual waveforms. In turn, the physiological assessment processor 340 may be configured to identify at least one characteristic feature for at least two of the individual PPG waveforms, wherein the at least two of the PPG waveforms are associated with differing distinct electromagnetic wavelengths.

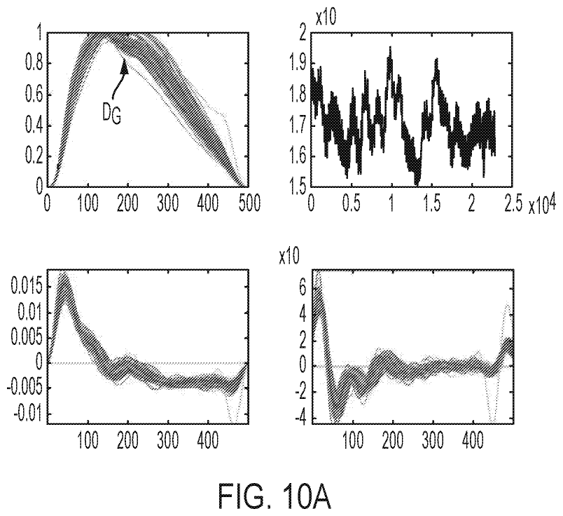

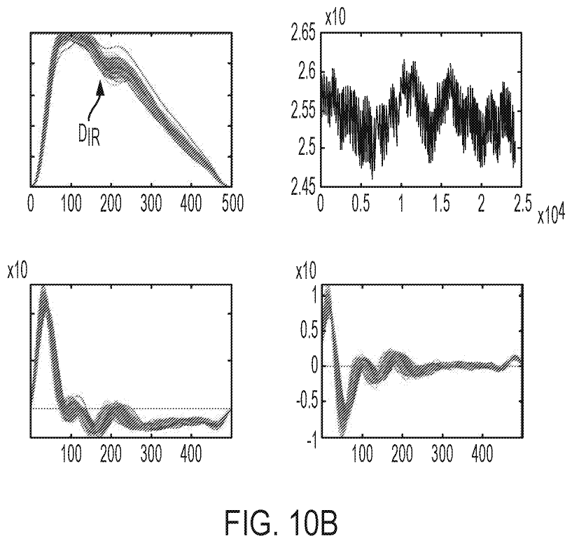

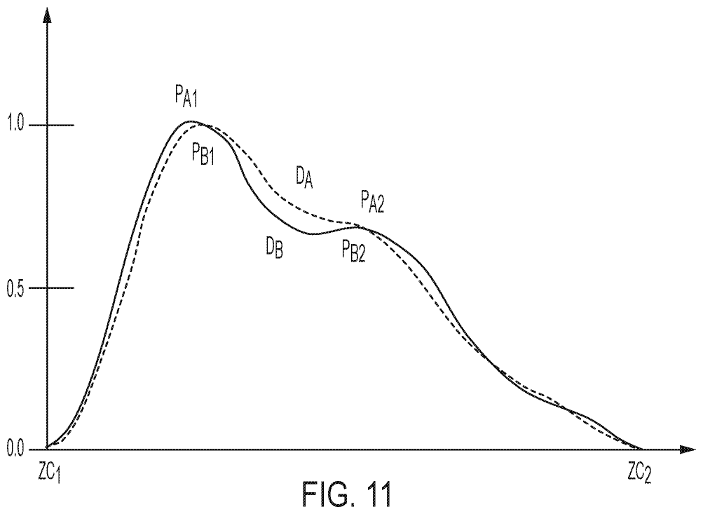

There are many ways to utilize this innovation, and one example is illustrated in FIGS. 10A-10B. FIGS. 10A-10B illustrate raw PPG waveform data taken from the same subject at the same body location using two distinct electromagnetic wavelengths in the green (˜520 nm) and IR (˜940 nm) ranges, showing a notable difference between the dicrotic notch (D) of each wavelength. Namely, the dicrotic notch features of the IR wavelength are notably more pronounced than those of the green wavelength. The waveform processor 330 may be configured to normalize and parameterize these two PPG datasets (in this case one IR and one green) and create average or median waveforms from the respective PPG datasets. For example, PA1, PA2, and DA may be parameters generated for the green PPG data (FIG. 11) and PB1, PB2, and DB may be parameters generated for the IR PPG data (FIG. 11), and these parameters may be processed as characteristic features for the respective PPG waveforms (IR and green). The physiological assessment logic of the assessment processor 340 may be configured to process at least one characteristic feature from each of the two PPG waveforms (IR and green). As a specific example, the difference between the DA and DB feature may be related to the blood pressure of the subject. Although just two wavelengths were provided in this example, the invention could employ a plurality of PPG waveforms over a plurality of optical wavelengths beyond just two.

It should be noted that other characteristic features of a physiological waveform may be processed by the waveform processor 330. As a specific example, key time intervals in an ECG waveform may be processed as characteristic features of an ECG. Examples of such time intervals include, but are not limited to, the P-R interval, QRS interval, Q-T interval, S-T interval, and the like. As another example, statistics in the time intervals between a plurality of consecutive ECG waveforms or PPG waveforms (such as the HRV statistics in a set of RR-intervals or PP-intervals respectively) may be processed as characteristic features. Additionally, features of transforms of a group of consecutive biometric waveforms may be processed as characteristic features. As specific example, a group of consecutive PPG waveforms may be transformed into the spectral domain, and the resulting spectral features may be processed as characteristic features.

### c) Enabling a Relationship Engine

The biometric waveform analysis system 10 of FIG. 2 and FIG. 3 may be configured to generate a relationship between the extracted physiological parameters and the physiological waveforms. This functionality may be executed primarily by the relationship processor 500. A key benefit of generating such a relationship is that it may be less processor intensive to generate a high-rate metric output of a physiological parameter continuously rather than to generate a waveform assessment continuously. The biometric waveform analysis system 10 may comprise relationship logic configured to generate a relationship between the extracted at least one physiological parameter (or motion parameter) and the individual physiological waveforms (e.g., the individual physiological waveform assessments). The derived relationship may then be integrated into the metric output generator 200 such that the relationship may be executed in real-time, continuously, at a high rate rather than requiring a longer period of processing time or greater processing requirements, as may be required to process a PPG waveform or a group of PPG waveforms.

Phrased another way, the relationship engine is configured to create a transfer function between the extracted physiological parameters and the physiological waveform assessments, whereby the inputs to the transfer function are measured physiological parameters and the outputs are estimations of the physiological waveform assessments. Thereby the transfer function may then be integrated into the metric output generator 200 such that the transfer function may be executed in real-time, continuously, at a high rate rather than requiring a longer period of processing time or greater processing requirements.

As one specific example, the relationship logic of the relationship processor 500 may be configured to generate a relationship between an extracted motion parameter (extracted from the motion sensor signal 110) and a physiological waveform (extracted from a physiological sensor 110). For example, the high rate calculation of user cadence (the frequency of a user activity, such as step rate, cycling rate, etc.) may be generated by the metric output generator 200, and the waveform analysis engine 300 may process the ECG waveform data to generate heart rate data or PQRST parameters (such as time between features these features or the relative magnitudes of these features, such as PR-interval, QRS complex features, ST segment features, and the like). The relationship processor 500 may then process the time-correlated cadence data and the processed ECG waveform data to generate a relationship between user cadence and heart rate or user cadence and PQRST parameters. This derived relationship may then be integrated into the metric output generator 200 such that continuous real-time estimations of heart rate and PQRST parameters may be generated using the cadence sensor alone. Thus, this invention may save processing power (as ECG waveform calculations may be minimized) and improve monitoring robustness (as, during physical activity, calculating user cadence is inherently more robust than is measuring voltage from a wearable electrode). Moreover, methods of calculating user cadence and ECG parameters are well-established and well-known to those skilled in the art. (Note: In a similar example, cadence may be replaced by another motion parameter, such as speed, motion intensity, acceleration, and the like.) Additionally, once the derived relationship is developed, the ECG sensor may no longer be needed. Alternatively, the relationship may be autonomously augmented from time-to-time to keep the relationship up-to-date, as the transfer function between subject motion parameters and subject ECG waveforms may change over time.

Similarly, in another specific example, the relationship logic of the relationship processor 500 may be configured to generate a relationship between an extracted physiological parameter (extracted from a physiological sensor signal) and a physiological waveform (extracted from a physiological sensor). For example, a high-rate, motion-tolerant streaming PPG output may be generated by the metric output generator 200, and the waveform analysis engine 300 may process the ECG waveform data to generate the aforementioned PQRST parameters. The relationship processor 500 may then process the time-correlated motion-tolerant streaming PPG data and the processed ECG waveform data to generate a relationship between user PPG data and the PQRST parameters. This derived relationship may then be integrated into the metric output generator 200 such that continuous real-time estimations of PQRST parameters may be generated using the PPG sensor alone. It should be noted that the relationship processor 500 may alternatively generate a relationship between the streaming PPG data and streaming ECG data such that a relationship is developed mapping the PPG data to the ECG data. In such case, by simply wearing a PPG sensor 110, the metric output generator 200 may generate an ECG waveform estimation for the subject. Though potentially lower-acuity in nature, a PPG-derived ECG waveform estimation may be much more convenient to wear than ECG electrodes. The generation of this relationship between the PPG data and the ECG data may employ machine learning (neural network) technology to learn a relationship between the PPG waveforms and the ECG waveforms over time.

In the aforementioned examples of generating PPG-derived ECG via the biometric waveform analysis system 10 (i.e., via the relationship processor 500), it should be understood that the same basic process may be used to generate a streaming PPG-derived EEG waveform (i.e., streaming EEG) or to generate PPG-derived (or optical sensing based) EEG parameters. In turn, this enables a PPG-based method of monitoring brain function, cognition characteristics (such as cognitive load, alertness level, intent, drowsiness, allocation of mental resources, and the like), sleep characteristics, neurological functioning, and the like. Examples of EEG parameters known to those skilled in the art include entropy, WSMF, qWSMF, delta wave parameters, alpha wave parameters, beta wave parameters, theta wave parameters, approximate Lempel-Ziv complexity, voltage, morphology, frequency characteristics, synchrony, periodicity, and the like.

It should be noted that, for the example above, the source of the ECG data may be a continuous ECG sensor or a spot-check ECG sensor. Namely, depending on the location of the ECG device, it may be difficult to generate high-acuity continuous ECG signals that are good enough to provide useful PQRST parameters. For example, the wrist location is a poor location for ECG. Although ECG and bioimpedance may be measured, the signal quality for continuous monitoring (i.e., with two electrodes on the wrist) is typically very weak and often not useful. Unfortunately, depending on a variety or subject-dependent factors, the only suitable place for continuously monitoring high-acuity ECG may be on the torso, as with an ECG chest strap, and ECG patch, or other ECG electrode configuration. Thus, a spot check may be required for high-acuity ECG on non-torso form-factors. For example, an ECG electrode configuration on a wrist watch, with at least one electrode against the wrist and at least one electrode against an opposing finger of the subject to complete the ECG circuit, may allow an acute spot check of ECG for the subject, while PPG wrist sensors are continuously measuring PPG information from the subject. Such ECG electrode configurations in wrist watches, where the subject is instructed to place a finger on an electrode on the watch (with an underlying electrode in contact with the skin of the subject's wrist), are well known to those skilled in the art. A similar configuration may be used for digit-worn (e.g., finger-worn or toe-worn) ring device, with at least one electrode against the digit and at least one electrode on top of the ring such that an opposing finger of the subject can be used to complete the ECG circuit. (See FIG. 27.)

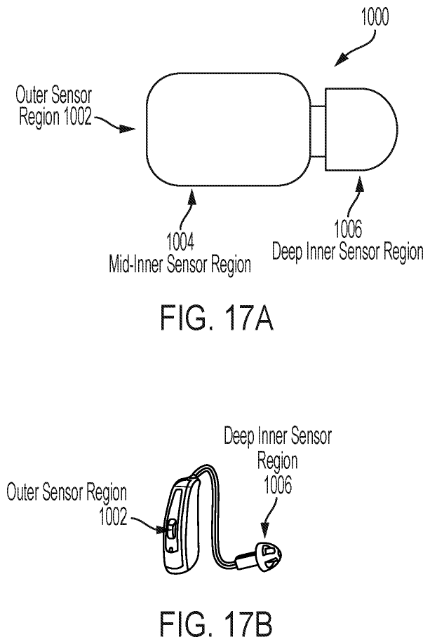

Alternatively, in a novel embodiment, the subject may be instructed to place their finger across at least one electrode, or other sensor, in an earpiece 1000; an example of such a novel ECG electrode configuration integrated into an earpiece 1000 is illustrated in FIG. 17A. Three electrodes are described: an outer sensor region 1002, a mid sensor region 1004, and a deep sensor region 1006. The mid and deep sensor regions 1004, 1006 may be in intimate contact with the mid and deep ear canal regions of the subject, respectively. In contrast, the outer sensor region 1002 may be exposed and assessable by a finger press, thereby completing the ECG circuit and enabling a high-acuity ECG reading for the subject. While it is true that an ECG reading may be generated across the mid and deep electrodes 1004, 1006, the signal may be very weak, as the potential drop across the two electrodes may be extremely small. However, the potential drop between the finger of the subject and the mid and/or deep regions of the ear canal may be quite substantial and straightforward to detect. It should be noted that the sensor regions shown in FIG. 17A should not be limited to ECG sensors for all sensing embodiments. For example, the sensor regions may also be PPG sensors, EEG sensors, bioimpedance sensors, combinations of various sensors, or the like. For example, in one embodiment, at least one of the mid or deep sensor regions 1004, 1006 is an ECG electrode and the other is a PPG sensor, and the outer sensor region 1002 is an ECG sensor. In such case, the time difference between the ECG signal detected via the outer sensor region 1002 (with the mid and/or deep sensor region comprising a reference electrode) and the responsive PPG signal detected via the PPG sensor of the mid or deep senor region 1004, 1006 may be used to assess (i.e., via the assessment processor 340) the pulse wave velocity (PWV), pulse transit time, and/or vascular elasticity for the subject. This is because the time difference between the ECG signal and the resulting blood flow pulse may be inversely related to blood flow speed, inversely related to arterial stiffness, and directly related to vascular health. A similar outcome may be generated by the outer sensor region 1002 being a PPG sensor 110, and monitoring the time difference between the finger PPG signal and the ear canal PPG signal.

The innovative biometric waveform analysis system 10 enables a novel method of generating a continuous physiological assessment for a subject, wherein the subject is wearing a PPG sensor 110 that generates a PPG data signal comprising high-rate PPG information (such as information related to heart rate, breathing rate, blood perfusion, pulse volume, PPG spectral magnitude (at the heart rate frequency, PPG pulsatile maxima and minima amplitudes in the time-domain, and the like) and PPG waveform information, the method comprising: sampling a plurality of PPG waveforms from the PPG signal; generating at least one high-rate physiological metric from the PPG signal; generating a high-acuity physiological assessment of the subject from the PPG waveforms, over a time interval, by processing the PPG waveforms via the at least one waveform analysis engine 300, wherein the at least one high-rate physiological metric is mapped to the high-acuity assessment over the time interval; and outputting a continuous physiological assessment for the subject, wherein at least some physiological assessment values are based on a mapped relationship between the at least one high-rate physiological metric and the high-acuity physiological assessment.

As a specific example of the above, the high-rate PPG metrics generated by the metric output generator 200 may comprise heart rate information and/or pulse volume information. (Just as an example, generating a continuous biometric parameter of heart rate may be achieved by real-time pulse picking over peaks or troughs of a noise-filtered PPG signal. Generating a continuous biometric parameter of blood pulse volume may be generated by processing the PPG signal in real time to determine the real-time spectral amplitude at the heart rate frequency, which may be proportional to blood pulse volume.) The high-acuity physiological assessment generated by the waveform analysis engine 300 may comprise blood pressure. (Just as an example, generating a blood pressure assessment may comprise processing waveform features over a plurality of PPG waveforms using a machine learning model.) Generating a relationship (i.e., via the relationship processor 500) between a high-rate metric (i.e., pulse rate and/or pulse volume) and the high-acuity assessment (blood pressure) may help reduce processing resources required to generate a continuous blood pressure assessment. Namely, the mapped relationship between the high-rate physiological metric and the high-acuity physiological assessment of blood pressure may be processed more efficiently in real-time (via the metric output generator 200) than processing a plurality of PPG waveforms (via the waveform analysis engine 300) to generate a blood pressure values. Although the acuity of the blood pressure assessment may be lower, the processing resources may be so substantially reduced that the short-cut may be warranted. Moreover, as the mapped relationship may diverge over time, perhaps due to physiological changes in the subject over time, the relationship processor 500 may be configured to update the relationship over time based on a fixed interval, random interval, or an interval smartly (autonomously) chosen by processing sensor signals in context of system confidence indicators.

In another embodiment, the innovative biometric waveform analysis system 10 enables a novel method of mapping at least one low-power high-rate metric to high-power high-rate metric, such that a lower-power assessment can be made using the low-power high-rate metric alone, with sufficient acuity. As a specific example, signals from a PPG sensor 110 may be processed by the metric output generator 200, such that at least two metrics are generated, such as real-time heart rate (a lower-power calculation) and real-time RRi (a higher-power calculation). The relationship processor 500 may then process, over a time interval, the at least two metrics such that the lower-power physiological metric (heart rate) is mapped to the higher-power physiological metric (RRi) over the time interval; and this relationship may then be programmed into the metric output generator 200 (e.g., autonomously via the control processor 400) such that a new high-rate metric output is generated that comprises a lower-power estimation of RRi. Thus, the real-time calculation of RRi, which may be using sampling frequencies>250 Hz, may be turned-off for the subject following the said time interval. This innovation can be extremely useful for HRV-based stress monitoring (achieved by generating statistics on a series of consecutive RR-intervals) in a wearable device, as monitoring stress continuously in real-time may require high-speed processing of the PPG (or ECG) waveform to generate high-acuity RRi. In contrast, processing high-acuity heart rate may require substantially less processing resources (i.e., as much as 10× less sampling frequency) than that of high-acuity RRi. Thus, a method of, over a time interval, enabling a wearable device to learn a lower-power estimate of RRi, based on HR alone, can be extremely useful by enabling continuous stress monitoring with improved (i.e., extended) battery life. Nonlimiting examples of processing RRi information to generate stress assessments may be found in U.S. Application Publication No. 2018/0220901, which is incorporated herein by reference in its entirety.

Moreover, building this relationship requires no input from the user, as it is completely autonomous. It should be noted that this example was provided using only heart rate as the lower-power metric; however, mapping additional lower-power metrics (heart rate+other lower-power metrics) to RRi may improve the acuity of the RRi estimate. For example, to generate high-acuity heart rate from a 25 Hz sampled PPG signal, it may be necessary to process metrics of peak spectral amplitude, DC baseline, and the like. These additional PPG metrics are generated computationally “for free” in the required processing of motion-tolerant heart rate (as described in U.S. Pat. Nos. 10,349,844; 9,993,204; 9,801,552; and 9,794,653; which are incorporated herein by reference in their entireties). Thus, adding them to the estimation or RRi requires no significant power burden and may improve the accuracy of the estimation. Additionally, adding accelerometer information or motion-related parameters (such as user cadence, speed, pace, and the like) to the relationship engine mapping may also improve the accuracy of the estimate of RRi. One particularly novel and useful aspect of this invention is that complex high-power PPG metrics (such as blood pressure and RRi) may be mapped to streaming, low-power PPG parameters or metrics (such as PPG maxima and minima amplitude or heart rate) using the very same PPG sensor (a single PPG sensor).

Indeed, in the above-mentioned examples, the heart rate and RRi estimation were generated using the same PPG sensor. However, this innovation is not limited to PPG sensors alone. In principle, a plurality of sensors may be used. For example, in one embodiment, such as with an ear-worn or head-worn wearable, the high-power high-rate metric may comprise EEG, and the low-power high-rate metric may comprise at least one PPG metric/parameter and at least one motion metric/parameter (but preferably a plurality of PPG metrics/parameters and a plurality of motion metrics/parameters, as well as raw accelerometry data). The benefit of this innovation is not only power savings but also an improvement to long-term wearability. Namely, wearing contact electrodes (as required for EEG) over a long period of time is simply not comfortable for human subjects (or any animal for that matter). But PPG sensors are much more agreeable for long-term wear. Thus, a mapped relationship between EEG metrics and PPG metrics can be highly functional, such that (in the long-term) a PPG-only solution may be of sufficient acuity in estimating EEG for the subject, thereby improving power management of the wearable as well as long-term comfort for the subject. As a specific example, the heart rate variability (HRV) of a subject, as determined by processing consecutive the PPG-derived RR-intervals from the subject, may be correlated with EEG assessments of cognition. Additionally the rising and falling slopes of the PPG waveform may also correlate with EEG-based assessments. In building sufficient data for the relationship engine to create a map between EEG and PPG parameters/features, the subject may need to wear an EEG device and a PPG device for a sufficient period of time to generate a PPG algorithm of sufficient acuity to accurately estimate EEG parameters for the subject.

In another embodiment, the innovative biometric waveform analysis system 10 enables a novel method of mapping at least one low-power high-rate metric to high-power low-rate metric, such that a lower-power assessment can be made using the low-power high-rate metric alone, with sufficient acuity. This embodiment may be particularly useful when the sensor system 100 comprises a blood pressure monitoring cuff, a biochemical blood-draw sensor (e.g., sensors for blood panel readings of glucose, cholesterol, inflammation markers, and the like), an interstitial fluid sensor, saliva sampling sensor, a urine sensor, or other low-rate metric sensors that cannot be used for true continuous monitoring. As a specific example, signals from a PPG sensor 110 may be processed by the metric output generator 200, such that a plurality of PPG parameters are generated continuously in time. The relationship processor 500 may then process, over a time interval, the PPG parameters to map them to blood pressure cuff readings taken over that same time interval (but taken non-continuously, at a low-rate, due to inherent limitations in cuff-based BP monitoring). The mapped relationship (e.g., the transfer function) can then be programmed (e.g., autonomously, via the control processor 400) into the metric output generator such that a high-rate BP estimation can be generated continuously based on high-rate PPG parameters, without the need for additional BP cuff readings. Alternatively, this mapped relationship may be augmented with additional PG cuff readings over time, as the transfer function between PPG parameters and BP cuff readings may change over time.

It should be noted that the innovation above may be configured to generate relationships between a variety of high-rate metrics, high-acuity assessments, and continuous physiological assessments. For example, the high-rate metric may comprise blood pulse volume and heart rate information, the high-acuity assessment may comprise cardiac output, and the continuous physiological assessment may comprise cardiac output. As another example, the high-rate metric may comprise blood pulse volume information, the high-acuity assessment may comprise an assessment of volumetric blood flow, and the continuous physiological assessment may comprise volumetric blood flow information. Other metrics and assessments may be generated and utilized via this invention.

d) High-Rate vs. Waveform Processing

The values of high-rate metrics generated by the metric output generator 200 may be used to trigger processing in other parts of the biometric waveform analysis system 10. For example, the biometric waveform analysis system 10 may be configured wherein a physiological assessment of the subject is generated by the physiological assessment logic (i.e., within the assessment processor 340) in response to the at least one physiological parameter extracted by the metric output generator 200 and/or the at least one motion parameter extracted by the metric output generator 200. As a specific example, the at least one physiological parameter extracted by the metric output generator 200 may comprise a heart rate value, and the physiological assessment logic may be configured to generate an assessment of subject blood pressure if it is determined that the heart rate value is above or below a threshold. Such logic may save processing power by triggering a blood pressure reading only when it's likely that a blood pressure reading is warranted, as when the subject's heart rate is notably high or notably low. As another specific example, the at least one physiological parameter extracted by the metric output generator 200 may be a heart rate value, and the physiological assessment logic may be configured to generate an assessment of subject cardiac output or volumetric blood flow if it is determined that a heart rate value is above or below a threshold.

It should be noted that this utility may also be provided by processing motion parameter data (as with physiological parameter data) from the metric output generator 200. For example, the assessment processor 340 may be configured to generate a subject blood pressure assessment if the physiological assessment logic determines that the value of a motion parameter is above or below a threshold. As another example, the biometric waveform analysis system 10 may be configured such that, if the motion parameter extracted by the metric output generator 200 is on one side of a threshold, the physiological assessment of the subject generated by the physiological assessment logic is an assessment of subject blood pressure based on subject blood pulse volume, and wherein, if the motion parameter is on an opposite side of the threshold, the physiological assessment of the subject generated by the physiological assessment logic is an assessment of subject blood pressure based on a waveform parameterization. As a specific example, the assessment logic may determine that a user cadence (generated by the metric output generator) is below a safe value with sufficiently low motion artifacts such that calculating blood pressure via waveform parameterization is acceptable. However, if the cadence goes above the safe value, the assessment logic may switch to assessing blood pressure via an algorithm based on blood pulse volume (which may be generated continuously by the metric output generator as described earlier). More generally, it may be desirable for the waveform analysis system 10 to select the method of generating a subject's blood pressure assessment contingent on the activity state of the user.

### e) Actuators

The biometric waveform analysis system 10 of FIG. 2 and FIG. 3 enables a smart system of wearable actuators 600, which may be used for enhanced smart sensing. For the biometric waveform analysis system 10, a wearable device may comprise at least one actuator 600 configured to adjust the stability of the wearable device relative to the subject body in response to one or more of the following: a physiological assessment of the subject, a physiological parameter (i.e., as extracted by the metric output generator 200), a motion parameter (i.e., as extracted by the metric output generator 200), a sensor output, or the like. Apparatuses and methods that enable wearable systems to actuate in response to motion and physiological changes have been previously described in U.S. Patent Application Publication No. 2017/0119314, which is incorporated herein by reference in its entirety. However, new innovations herein describe apparatuses and methods that leverage the biometric waveform analysis system.

As one example, the biometric analysis system 10 may comprise: 1) at least one physiological sensor 110 that is an acoustic sensor (i.e., a sensor configured to sense sound-related vibrations) configured to obtain auscultatory sounds from the body of the subject, 2) at least one actuator 600 configured to cause a wearable device to create an acoustic seal with the body of the subject, such that a cavity is created that is in acoustic communication with the acoustic sensor. FIG. 15A illustrates an example of an earpiece 100, configured to be inserted within the ear canal EC of a subject, where in the earpiece 1100 comprises actuators 600 to create an acoustic seal at the front (closer to the tympanic membrane) of the earpiece and/or the back (farther from the tympanic membrane) of the earpiece 1100. It has been found through experimentation that integrating an acoustic sensor within the earpiece 1100, coupled to the sealed cavity, enables high-acuity auscultatory monitoring, enabling the detection or cardiac sounds, breathing sounds, digestive sounds, swallowing sounds, general mouth motion sounds, footstep sounds, general body motion sounds, and muscle-motion sounds from ear muscles and/or eye muscles. However, continual processing of acoustic sounds for high-quality auscultatory monitoring can be extremely processor intensive.

Moreover, a continual acoustic seal for the user can be uncomfortable for long-term wear. Thus, a key benefit of this invention is for the biometric waveform analysis system 10 to trigger the actuation of the acoustic seal (i.e., via the control processor 400) in response to a physiological assessment of the subject generated via sensor outputs from a lower-power, more comfortable sensor. As a specific example, the PPG signal from a PPG sensor 110 embedded within the earpiece 1100 may be processed to determine the breathing rate of the subject. The assessment processor 340 may assess (via processing the PPG sensor data) that the subject is having difficulty breathing or is at the onset of breathing difficulties. In response, the control processor 400 may send an activation signal to at least one actuator 600 in the earpiece 1100 to create at least one acoustic cavity, as shown in FIG. 15A. The assessment processor 340 may then process signals from the acoustic sensor 110 (coupled to the acoustic cavity) to monitor auscultatory waveforms for breathing sounds associated with a breathing difficulty (such as breathing volume abnormalities or erratic respiration rates). In this manner, the assessment processor 340 can make a higher acuity assessment of the breathing condition of the subject without burning too much battery power and without burdening the user with a continuous acoustic seal.

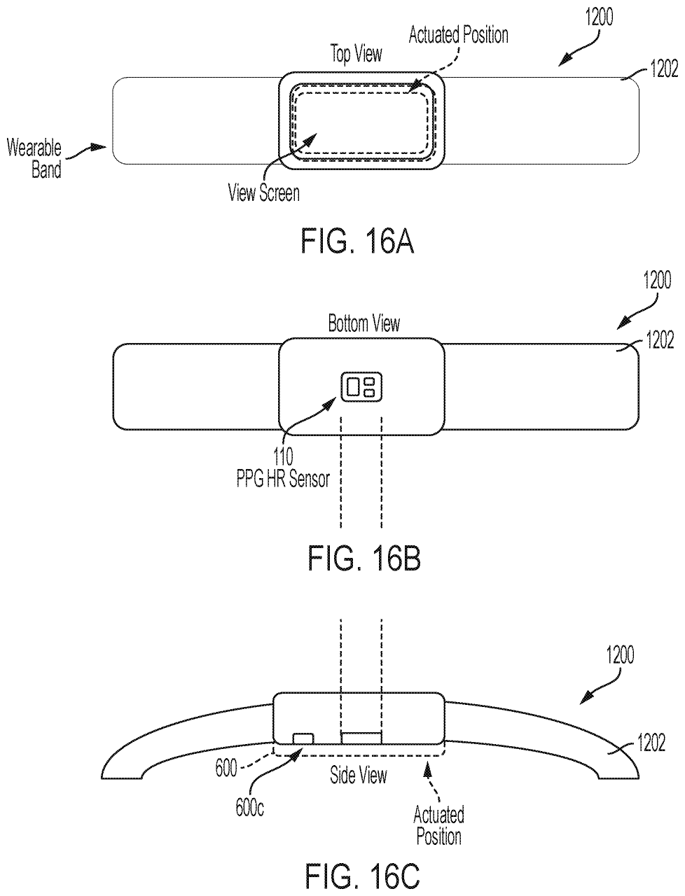

This innovation may also be provided for wrist-based monitors, where the actuator is configured to create an acoustic cavity between the wrist-based device 1200 and the wrist of the subject, as shown in FIGS. 16A-16C. As a specific example, the PPG signal from a PPG sensor 110 embedded within the wrist device 1200 may be processed to determine the blood pressure of the subject. The assessment processor 340 may assess (via processing the PPG sensor data) that the subject is experiencing abnormally high or abnormally low blood pressure. In response, the control processor 400 may send an activation signal to at least one actuator 600 (FIG. 16C) in the wrist-based monitor 1200 to create an acoustic cavity 600c between the wrist device 1200 and the wrist of the subject. The assessment processor 340 may then process signals from the acoustic sensor 110 (coupled to the acoustic cavity) to monitor auscultatory waveforms for blood pressure sounds associated with abnormally high or abnormally low blood pressure. In this manner, the assessment processor 340 can make a higher acuity assessment of the blood pressure status of the subject without burning too much battery power and without burdening the user with a continuous acoustic seal at the wrist of the subject. In this particular example, the actuation of an acoustic cavity may be replaced with the actuation of oscillometric coupling (as with an inflatable cuff around an appendage for blood pressure monitoring) and the auscultatory monitoring step may be replaced with an oscillometric monitoring step.

It should be noted that although the earpiece 1100 (FIG. 15A) and wrist-based device 1200 (FIGS. 12A-12C) are illustrated as examples for this innovation, other form-factors (such as armbands, legbands, patches, rings, neckbands, headbands, clothing, and the like) may also employ this invention, with respective seals between the body of the subject and the respective form-factor. At least one acoustic sensor is coupled to the acoustic cavity 600c and at least one alternate sensing mechanism (such as a PPG sensor or lower-acuity acoustic sensing mechanism) is used for screening for the need to activate the actuator 600. Additionally, the physiological assessments and associated form-factors are not limited to the specific descriptions herein. Namely, the assessment of breathing difficulties is not limited to the earpiece 1100 and the assessment of blood pressure status is not limited to the wrist-based device 1200. For example, the aforementioned example of using a wrist sensor for blood pressure monitoring can be equivalently applied to the earpiece 1100 of FIG. 15A, and indeed, an earpiece may be more accurate for assessing blood pressure via an acoustic cavity than would a wrist-based device.

For the aforementioned examples for the earpiece 1100 and wrist-based device 1200, where an actuator 600 is activated based to create an audio cavity, a variety of mechanisms may be used to generate the actuation. Nonlimiting examples can be found in U.S. Patent Application Publication No. 2017/0119314, which is incorporated herein by reference in its entirety. As just one example, a piezoelectric or inductive actuator, such as a MEMS (microelectromechanical system) actuator, may be employed to create the seal required to generate the acoustic cavity 600c. Because the earpiece 1100 is supported by the ear canal EC, and because the wrist-based device 1200 is supported by a band 1202 around the wrist, the actuation force may be applied outwardly from the respective devices, with reactive forces from the ear canal EC and wrist (via the wrist band 1202), respectively, working together with the respective outward force to complete the seal. The seal within the ear canal EC should ideally be circumferential to create a seal within the wall of the ear canal EC, and the seal along the wrist should create a perimeter along the wrist to create a seal between the wrist and the wrist-facing portion of the wrist-based device. (See FIG. 15a and FIGS. 16A-16C.)

In another example of actuators 600 being used within the biometric waveform analysis system 10, the actuator 600 may be an electromagnetic emitter, configured to stimulate a region of the body of a subject via electromagnetic radiation. A specific example, where the stimulating electromagnetic emitter comprises a stimulating optical emitter 602, is presented in FIG. 15B. The illustrated earpiece 1100 includes one or more optical emitters 602. Three preferred optical emission patterns 604 of the actuated optical emitter are presented in FIG. 15B. Two of the three emission patterns 604 are configured to stimulate the walls of the ear canal EC, and to penetrate through those regions, whereas the other emission pattern is configured to penetrate the quasi-transparent tympanic membrane of the subject. Unlike PPG optical emitters, which are configured to generate an optical scatter source for PPG sensing, the optical emitters 602 of FIG. 15B are configured for stimulation, not detection. Namely, the optical beams generated by these emitters are configured to stimulate a physiological outcome in the body of the subject. Three non-limiting examples of optical stimulation outcomes may include: 1) optical stimulation of the vagus nerve of the subject, 2) optical stimulation of the skin and neighboring tissues to stimulate localized blood perfusion for the subject, and 3) optical stimulation of the brain or eye muscles of the subject to generate and/or monitor neural or muscular activity therein. The optical wavelengths used to stimulate physiological outcomes may be virtually any optical wavelength, but for the two examples above, infrared optical wavelengths may be preferred for two reasons: 1) the penetration depth of infrared wavelengths is deeper than that of visible wavelengths and 2) infrared wavelengths are generally (though not always) more effective at heating and/or stimulating tissue than shorter wavelengths.

When combined with the sensing innovations of the biometric waveform analysis system 10, the actuation of optical emitters 602 for stimulating physiological outcomes can be autonomous and within a feedback loop, as needed by the subject. For example, the biometric waveform analysis system 10 may generate a stress assessment for the subject (i.e., using one of the methods for stress analysis described herein). Upon determining that the stress condition of the subject is problematic for the subject (i.e., by determining that the HRV pattern for the subject, as determined by processing statistics on consecutive RR-intervals, shows an overactive sympathetic nervous system response for the subject), at least one of the optical emitters 602 of FIG. 15B may be actuated to stimulate the vagus nerve of the subject. This, in turn, may stimulate the vagus nerve to relieve the stress condition of the subject (i.e., to stimulate a parasympathetic response for the subject). In addition, the effects of the optical dosing can be monitored in real time by the continuous monitoring of the stress condition of the subject via the biometric waveform analysis system 10. The determination of a stress condition for the subject may also involve processing heart rate and/or blood pressure for the subject to determine if these physiological parameters are above or below an appropriate range for the subject.

Similarly, considering the case of optical perfusion stimulation using the actuated optical emitter, a feedback loop can be used to control the stimulation. For example, the assessment processor 340 (FIG. 3) may determine that the subject's blood perfusion is excessively low. In one nonlimiting example, the determination of excessively low blood perfusion may be determined by identifying that the wearable device is being worn by the subject and then monitoring the PPG signal quality, using methods described in U.S. Pat. No. 9,794,653 and U.S. Patent Application Publication No. 2018/0353134, which are incorporated herein in their entireties, to sense poor signal quality despite the device being worn correctly—suggesting low localized blood flow for the subject. Once a status of low blood perfusion is determined, the control processor 400 may actuate at least one of the optical emitters 602 of FIG. 15B to stimulate increased blood perfusion. The assessment processor 340 may then continue to monitor the blood perfusion of the subject, and upon determining that sufficient blood perfusion exists, de-activate the optical emitter 602. If low perfusion is detected yet again, the optical emitter 602 may be actuated again, and so forth.

### f) Fall Prevention and Detection

The invention may also be applied towards predicting the onset of a fall for a subject, assigning a probability that a subject might fall, and/or detecting that a fall has occurred for the subject.

As a specific example, the assessment processor 340 may comprise logic configured to analyze the motion data signal from at least one of the motion sensors 110 and to make an estimation as to whether the subject has fallen down, and to generate an output parameter indicating the estimation. This processing may comprise, for example, processing multi-axis accelerometer signals to determine the position or movement of the subject over time. A probability may be generated for the probability that the subject is falling or has fallen, based on the pattern of motion derived from the motion sensor data. A variety of motion sensor processing methods for determining that someone is falling or has fallen are well known to those skilled in the art. As an innovation provided by the invention herein, the assessment processor 340 may comprise logic configured to analyze the physiological data signal from at least one of the physiological sensors 110 and to make an estimation as to whether the subject has fallen down, and to generate an output parameter indicating the estimation. This type of assessment may be generated following a determination that the subject is falling or has fallen by processing the motion sensor output, as described above. This processing of physiological data may comprise, for example, processing vital signs data to determine if the pattern is consistent with someone having fallen down. For example, someone lying down will typically have a blood pressure that is notably lower than when they are seated or standing. Thus, the assessment processor 340 may process sensor signals (such as PPG sensor signals) to determine that the subject's blood pressure is substantially lower than typical, and this may generate a higher probability that the subject has fallen. Alternatively or additionally, breathing may be monitored by a wearable breathing sensor 110, such as a wearable PPG sensor or wearable auscultatory sensor, such as an acoustic sensor configured to measure body sounds, and the assessment processor 340 may process data from a breathing sensor to determine that the subject's breathing has substantially changed. This may be indicative that the subject has fallen down. In particular, the assessment processor 340 may determine that the subject has moved from periodic breathing to erratic breathing, or vice versa, and this may be indicative that the subject has fallen. A variety of processing methods may be used for determining whether the breathing signals (or other motion or physiological signals) are periodic or erratic (non-periodic), and a few examples are provided in U.S. Patent Application Publication Nos. 2017/0112447 and 2018/0220901, which are incorporated herein by reference, in their entireties.

By combining motion signal information with physiological signal information, a more accurate probability may be generated to determine if the subject is at risk of imminent fall or has fallen. In such case, the motion signal data may be processed to determine that the subject has likely fallen down, and in response, the physiological signal may be processed to confirm whether or not the subject has fallen down. For example, if the motion sensor signals have been processed to determine that the pattern of motion is associated with falling, the physiological data may then be processed to determine if the vital signs have changed in such a way that the pattern is indicative of a fall. For example, if the heart rate data is dropping substantially following the determination of a high probability of a fall, the assessment processor 340 may determine that the person has indeed fallen. (It has been observed in Valencell's laboratories that moving from standing to laying down positions can reduce heart rate by more than 10 BPM in less than 1 minute.) Thus, the combined motion signal processing and physiological sensor signal processing can be used to help reduce false positives and negatives in determining that a subject has fallen. As one specific example, the assessment processor 340 may determine that a fall has taken place if it is determined that the subject has rapidly changed position from standing to lying down (i.e., by processing of a multi-axes accelerometer-based motion sensor 110) and, following this determination, determining that the subject's heart rate has changed by more than 10 BPM over the course of 20-60 seconds. Similarly, the determination of abnormal gait patterns plus abnormal vital signs patterns by the assessment processor may also be used to assign a probability that the person is likely to fall imminently. First processing motion signals (i.e., to determine if a fall is likely to happen or to have happened) to then conditionally trigger the processing of physiological signals (i.e., to determine the presence of abnormal vital signs patterns) may be particularly useful for saving power in the wearable device. This is largely because commercial accelerometers typically use much less battery power than do commercial physiological sensors, so turning on the physiological sensor 110 only when needed may be quite useful in extending battery life. The determination of abnormal vital signs by the assessment processor 340 may be generated via a variety of methods, including the methods described earlier (i.e., those described above with respect to the waveform classifier examples illustrated in FIGS. 5A-5C.)

As another specific example, for a subject wearing a wearable device having at least one motion sensor 110 and at least one acoustic sensor 110, the assessment processor 340 may determine that the subject has fallen down by: 1) first processing motion sensor signals to determine that the subject is likely falling down and 2) in response to determining it is likely that the subject is falling down, processing acoustic sensor signals and/or PPG sensor signals to identify that a sudden impact is happening or has happened. Internal Valencell studies have shown that a strong impact signal (comprising an intense broadband signal across a variety of spectral frequencies) is detected by acoustic sensors during an impact; a similar impact signal has been found to be detected by PPG sensors during an impact. This implementation for fall detection is particularly useful where acoustic or PPG sensor signals are automatically buffered (i.e., as controlled by the control processor) continuously or upon an indication that the subject is likely to be falling (as determined by processing signals from the motion sensor), with signal processing of the buffered samples being executed upon this indication that the subject is likely to be falling. This way, the acoustic and/or PPG sensor does not been to be turned on, and signals from these sensors do not need to be processed, unless the “subject falling” indication is made first. This can substantially save battery life for the wearable device. Continuous buffering may take up memory resources, but may not be as computationally intensive as continuous processing of the sensor data in the memory buffer. Thus, selectively controlling the processing of buffered data upon an indication that the subject is likely to be falling may substantially save power resources for wearable devices.

It should also be noted that in the above, an optical sensor 110 may be used as an alternative to an acoustic or PPG sensor 110, as the impact signal from a subject impact may be detected by an optical sensor 110 even if that sensor is not configured for PPG sensing. Using a PPG sensor 110 to detect an impact is particularly novel, as PPG sensors are configured to measure blood pulses, not impact signals, and indeed impact signals are highly undesirable for PPG sensing. Thus, when a PPG sensor is used for this purpose, separate filtering may be required for extracting the physiological information (i.e., heart rate, blood pressure, etc.) versus the motion information (i.e., the impact signal, body motion signal, etc.). One exemplary method of extracting both physiological and motion information from a PPG sensor is described in U.S. Pat. No. 8,923,941, incorporated herein by reference in its entirety, and the impact signal described herein may be derived by processing the motion information that is extracted by this method (or other methods). As just one example, a high-pass or high-frequency band-pass filter may be used to extract the impact signal from a PPG signal, as both physiological information and body motion information may be described by relatively narrow-band, low frequencies.

It should be noted that, for fall detection, a particularly important sensor for a wearable device according to embodiments of the present invention may be sensors configured to facilitate communication with the subject, such as a speaker and a microphone, VPU (voice processing unit), voice pick-up sensor or the like. Thus, if a fall has been detected, the subject can be reached audibly and be audibly monitored to determine the nature of the fall. In this embodiment, the biometric waveform analysis system 10 would ideally be connected to a remote system. For example, the wearable device may have a wireless bidirectional connection with a smartphone, with a second bidirectional wireless connection from the smartphone to the cloud. Alternatively, the wireless device may have a direct connection to the cloud. A fall may trigger a signal to a remote system, signifying that the subject must be reached audibly. The subject may then be reached via the speaker, and the subject may be able to communicate to the remote system via the microphone. In this embodiment, an earpiece (such as a hearing aid) may be particularly useful, as a voice sensor or bone conduction microphone within the earpiece may be able to pick up the voice of the subject via sensing mouth motions, even if the subject is not able to audibly speak. In such case, the assessment processor 340 may be configured to monitor and assess the voice information. Alternatively, a separate communication processing system may be used to monitor the voice information, independently of the biometric information.

### g) All-Purpose PPG Sensor

The functionality of the biometric waveform analysis system 10 may require sensor module configurations that are non-traditional in design. For example, FIGS. 12A-12B illustrate a PPG sensor 110 in a band-like device 1200 (FIGS. 12A-12B) and in an earpiece 1100 (FIG. 12C). It should be noted that the form-factors shown in FIGS. 12A-12C are exemplary only and not meant to be limiting. The band-like device 1200 may generally represent a wearable biometric sensor design adapted to be worn around an appendage, such as a wristband, wristwatch, armband, legband, ring (i.e., to be worn on a digit), or the like. The earpiece 1100 may represent an earbud, hearing aid, headphone, ear jewelry, headset, or the like. The emitter and detector spacings in the PPG sensor 110 are typically configured to be optimized for heart rate monitoring. In such a configuration, the emitter-detector spacing should be close enough to allow enough photons to be transmitted in and out of the skin region (such that a sufficient photon density can be received by the detector to provide sufficient resolution of the PPG waveforms), but far enough apart to capture a sufficient ratio of modulated photon flux transmitted through physiological pathways that have been modulated by pulsatile blood flow. Namely, if the spacing is too far then insufficient light may be sensed to generate meaningful signal, but if the spacing is too close then too much unwanted light—light that has not passed through a blood-flow modulated region—may be sensed. Thus, the ideal would be to generate the highest signal-to-noise ratio of pulsatile light modulation vs. noise light information. For example, even if just one bit of optical signal flips between 1 and 0 bits during a blood pulse, the signal to noise can be extremely high if the baseline detector value (without pulsating blood) is just 0 or 1 bits. However, for optimal PPG waveform processing, the resolution and dynamic range of the pulsatile signal may be more important than the aforementioned signal-to-noise ratio. This is because accurate waveform processing may require high-resolution waveform shape information, and there is no significant waveform shape information in just one bit of detector signal. Moreover, whereas accurate heart rate information is important during exercise, waveform processing—as used to estimate blood pressure from PPG waveforms—may not be critical during exercise. So, for the case of waveform processing, the background noise level (as long as it is stable during the subject's resting state) can be quite high, yet easily removed via filtering (such as via low-pass, high-pass, bandpass, notch or DC-blocking filtering, or the like). In such case, it may be desirable to reduce the spacing between the optical emitters and optical detectors for waveform processing, such that they are closer together than they would ideally be during heart rate monitoring, thereby increasing the resolution of the PPG waveform. This poses a challenge for the product designer, as the optimal optomechanics for heart rate monitoring may not be the same as the optimal optomechanics for waveform processing (i.e., processing PPG waveforms to estimate blood pressure, cardiac output, hemodynamics, and the like).

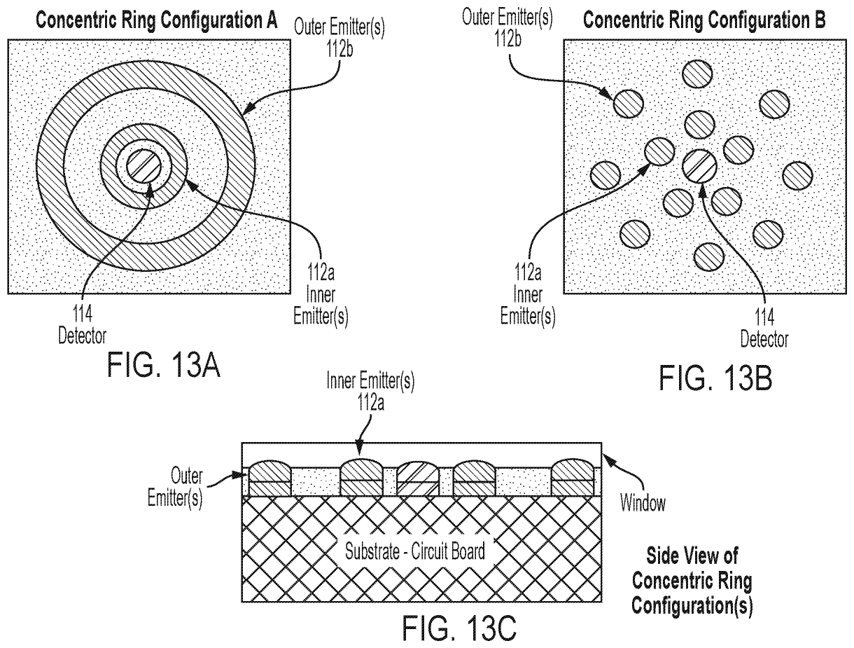

To address this engineering problem, a novel concentric ring optical configuration has been developed by the Applicant and is illustrated in FIGS. 13A-13C. In this configuration, the emitters are configured in two concentric rings around the optical detector 114. The first set of optical emitters 112a (the inner emitter ring) extend around the optical detector 114, and the second set of optical emitters 112b (the inner ring) are radially spaced apart from the first set of optical emitters 112a and extend around the first optical emitters 112a. The outer emitter ring is configured to generate an optical pathway used for monitoring periodic biometric parameters, such as heart rate, breathing rate, blood flow, and the like, whereas the inner ring is configured to generate an optical pathway used for monitoring waveform biometric parameters, such as blood pressure, blood analyte concentrations, cardiac conditions, and the like. The optical pathway generated by the outer ring of emitters 112b optimizes the signal-to-noise ratio, such that periodic biometric parameters can be accurately monitored during motion, and the optical pathway generated by the inner ring of emitters 112a optimizes the total PPG signal resolution, such that waveform biometric parameters can be accurately assessed (predominantly during resting conditions).

In practice, the biometric waveform analysis engine 300 (i.e., via the control processor 400) may be configured to alternately power on/off the inner and outer rings of emitters 112a, 112b such that the periods of turn-on time do not overlap. In such case, alternate samples for period biometric parameter processing and waveform biometric parameter processing may be generated for continuous processing of each sensing mode using one centralized optical detector.

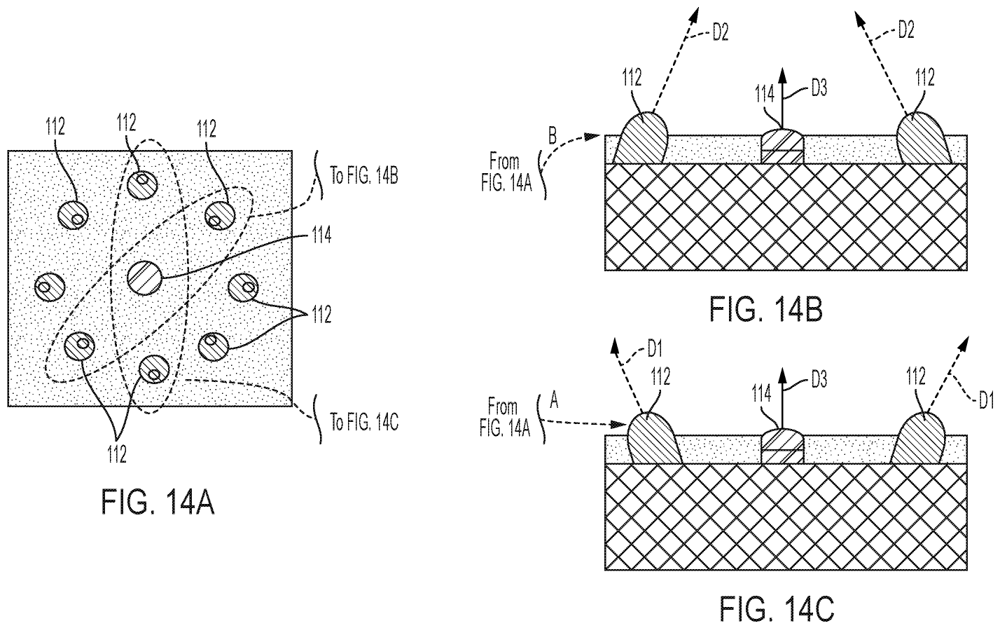

One aspect of the concentric ring configuration of FIGS. 13A-13C is that the total sensor area (emitter+detector+optics) may be larger than desired. This may be especially limiting for earpiece form-factors and finger (ring-worn) form-factors, where the wearable device is extremely small, with limited sensor real estate. To address this limitation, an alternate optical configuration may be desirable, such as that illustrated in FIGS. 14A-14C. FIGS. 14A-14C illustrate a plurality of optical emitters 112 positioned around the optical detector 114, wherein a first set A of the plurality of optical emitters 112 is oriented to emit light in a first direction D1, and wherein a second set B of the plurality of optical emitters 112 is oriented to emit light in a second direction D2, and wherein the first direction D1 extends outwardly from a direction D3 normal to a light receiving surface of the optical detector 114, and wherein the second direction D2 extends inwardly towards the direction D3 normal to the light receiving surface of the optical detector 114. This novel configuration enables optimization for both periodic biometric parameter monitoring as well as waveform biometric parameter monitoring, without the need for concentric rings of emitters. Instead, there is one ring of optical emitters 112 comprising alternating acute and obtuse light-guiding. Namely, some optical emitters 112 comprise light-guiding that direct light away from the detector 114—obtuse light guiding—and other optical emitters 112 direct light towards the optical detector 114—acute light guiding. The obtuse light guiding, emitting light in an outwardly direction, may be beneficial for monitoring periodic biometric parameters, as the total optical path is longer, thereby enabling a higher S/N (signal-to-noise ratio) of physiological signal to noise which can help with removing motion artifacts. In contrast, the acute light guiding, emitting light in an inwardly direction, may be beneficial for monitoring waveform biometric parameters, as the total physiological signal resolution collected by the detector may be much higher (even though the total DC noise may also be higher and the motion tolerance may be lower). As show in FIGS. 14A-14C, the plurality of optical emitters 112 is positioned around the optical detector 114 in a substantially equidistant, circumferential spaced-apart relationship to define a plurality of pairs of opposing emitters, and wherein each emitter 112 in a pair is oriented in the same first or second direction D1, D2. However, other configurations may be used within this invention.

Is should be noted that these novel optical configurations may comprise a plurality of optical emitters 112 emitting at a plurality of optical wavelengths. In addition, the invention may still be practiced by swapping optical detectors with optical emitters and optical emitters with optical detectors. Thus, the configuration of FIGS. 13A-13C is still valid, but the location of emitters and detectors are swapped. Namely, the critical element for enabling dual-optimization of monitoring periodic and waveform biometric parameters is for the optical pathways to be individually optimized, and this may be achieved with an outer ring of emitters 112 concentric with at least one optical detector 114 or with an outer ring of optical detectors 114 concentric with at least one optical emitter 112. Similarly, in an emitter/detector role-swap of FIGS. 14A-14C, light-guiding may be integrated into a plurality of optical detectors 114, wherein a first set of a plurality of optical detectors 114 is oriented to preferentially detect light from a first direction, and wherein a second set of the plurality of optical detectors 114 is oriented to preferentially detect light from a second direction, and wherein the first direction extends outwardly from a direction normal to a light emitting surface of an optical emitter 112, and wherein the second direction extends inwardly towards the direction normal to the light emitting surface of the optical emitter 112.

It should also be noted that in the above discussion regarding the differences in optimal directionality (for processing periodic biometric parameters vs. waveform biometric parameters), the beam patterns were assumed to be identical or highly similar in nature. Namely, the beam pattern of an inwardly facing optical beam and an outwardly facing optical beam was not specified, and may be assumed to be identical. However, in some wearable PPG configurations, the optimal beam pattern for monitoring heart rate may be a diffuse beam pattern (as opposed to a traditional linear beam pattern). At the same time, the optimal beam pattern for monitoring blood pressure may be a linear beam pattern (i.e., a focused or collimated beam pattern, as opposed to a diffuse beam pattern). The reasoning is that a diffuse beam pattern may be more stable during motion, where heart rate monitoring is particularly important. In contrast, a linear beam pattern, though less stable during motion, may be more efficient at transmitting photons towards the desired target. To accommodate both beam patterns, the inward-facing optics of FIGS. 13 and 14 may be configured to be traditional linear optics, whereas the outward-facing optics may be configured to be diffuse optics. A variety of methods well known to those skilled in the art may be employed to generate the diffuse pattern in the emitter or detector optics—for example, the diffuse pattern may be generated by roughening the surface of the optical lenses or light-guides, or by etching or depositing a pattern onto the surface of the optical lenses or light-guides. It should be noted that more generally, regardless of the directionality of the emitters or detectors in the PPG wearable, having at least some optics with beam patterns tailored for periodic biometric parameters and at least some optics with beam patterns tailored for waveform biometric parameters, within the same device, and with sequential sample collection from each of the two beam patterns, may be beneficial. For example, an array of emitter or detector optics that is facing directly towards the skin (as opposed to inwardly or outwardly) may be configured to have some optics diffuse and other optics not diffuse, such that data from each can be sampled sequentially in time.

For the embodiments shown in FIGS. 13A-13C and FIGS. 14A-14C, a variety of optical emitters and detectors may be used (as described earlier). In a preferred embodiment, the optical emitters are solid state optical emitters (such as LEDs or LDs) and the optical detectors are solid state optical detectors (such as photodiodes, phototransistors, photoconductors, or the like).

### h) Smart Sensing

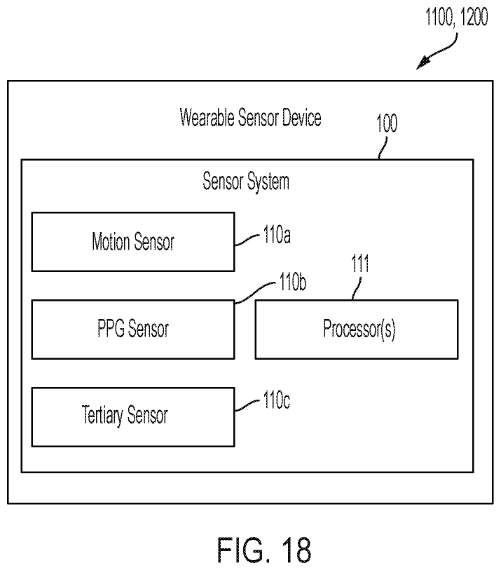

The biometric waveform analysis system 10 enables a variety of novel functionality, including a method of generating physiological assessments by smart sensing via contingent signal processing of a plurality of sensors 110. The method may comprise generating a physiological assessment for a subject via a sensor device (e.g., 1200, FIGS. 12A-12B; 1100, FIG. 12C) worn by the subject, wherein the sensor device comprises a motion sensor 110a, a PPG sensor 110b, a tertiary sensor 110c, and at least one processor 111, for example as illustrated in FIG. 18. The method may comrpise: 1) screening, via the at least one processor 111, a motion signal generated by the motion sensor 110a to determine if PPG monitoring of the subject is warranted, 2) in response to determining that PPG monitoring of the subject is warranted, screening, via the at least one processor 111, a PPG signal from the PPG sensor 110b to determine if monitoring of the subject via the tertiary sensor 110c is warranted, 3) in response to determining that the monitoring of the subject via the tertiary sensor 110c is warranted, screening, via the at least one processor 111, a signal from the tertiary sensor 110c, and 4) processing, via the at least one processor 111, the signal from the tertiary sensor 110c to generate a physiological assessment for the subject. A key benefit of this novel method may be power savings and extended battery life, as higher-power-consumption sensors may be used only when necessary. Additionally, some sensors—such as sensors which require actuation or user input, as described earlier—may create an inconvenience for the subject when utilized for too long of a period of time, and so continuous screening via other low-burdensome sensors (such as motion sensors or PPG sensors) may be used to determine when the user should be “inconvenienced” with such a tertiary sensor.

In one embodiment, the tertiary sensor 110c is an ECG sensor and the tertiary signal is an ECG signal. The physiological assessment may comprise an assessment of a cardiac condition of the subject, such as the presence of arrhythmia, heart pumping issue, or the like. In this embodiment, the processor 111 may process signals from the motion sensor 110a to determine when the subject is at rest or when motion artifacts would be expected to be low enough to enable a high-acuity PPG measurement. Under at least one of such circumstances, a PPG measurement may be determined to be warranted, and the processor may then execute processing of PPG signals to determine if the subject may be experiencing arrhythmia. Such processing may comprise analysis of consecutive RR-intervals, as determined by PPG, to screen for HRV patterns that are consistent with arrhythmia. Under such circumstances, a tertiary ECG sensor measurement may be warranted. The processor may then process ECG signals to generate an assessment of cardiac condition for the subject. Examples of cardiac conditions that may be generated by ECG analysis are well known to those skilled in the art. Depending on the wearable device configuration, a completely autonomous ECG signal may be generated without user involvement. However, in other cases, as described in context of FIG. 17A, following the determination that a tertiary ECG sensor measurement is warranted, the processer may instruct a communication (such as an audible or visual communication through the wearable device itself or a remote device in communication with the wearable device) to the subject that they place their finger on an ECG sensor located on the wearable device to complete the circuit and allow for an in-session ECG measurement to occur.

In one embodiment the tertiary sensor 110c is an auscultatory sensor and the tertiary signal is an auscultatory signal. The physiological assessment may comprise an assessment of breathing condition of the subject, such as that a respiratory condition (i.e., a particularly desirable or undesirable respiratory event) is imminent, or the like. In this embodiment, the processor 111 may process signals from the motion sensor 110a to determine if the subject is at rest or when motion artifacts would be expected to be low enough to enable a high-acuity PPG measurement. Under at least one of such circumstances, a PPG measurement may be determined to be warranted, and the processor 111 may then process PPG signals to determine if the subject may experience a breathing condition—such as problematic breathing, abnormal respiration rate, abnormally low blood oxygenation, substantially improved breathing, normal breathing or the like. Under at least one such circumstance, a tertiary auscultatory sensor measurement may be warranted. The processor 111 may then process auscultatory signals to generate an assessment of the breathing condition of the subject. This may be achieved by processing body sounds to identify sounds associated with problematic breathing, abnormal respiration rate, and the like. Thus, by using the PPG sensor 110b as a screen and then conditionally processing the auscultatory information only when needed, a more confident assessment of the breathing condition may be generated than by using PPG alone, but without continuously burning precious battery power to process auscultatory signals.

In one embodiment, the tertiary sensor 110c may be an optical sensor, such as an imaging sensor (i.e., a camera, optical sensor array, or the like) or a spectrometer (such as an optical spectrometer, Raman spectrometer, or the like). The physiological assessment may comprise an assessment of the cardiac or respiratory condition of the subject. In this embodiment, the processor 111 may process the motion sensor to determine if the subject is at rest or when motion artifacts would be expected to be low enough to enable a high-acuity PPG measurement. Under at least one of such circumstances, a PPG measurement may be determined to be warranted, and the processor 111 may then process PPG signals to determine if the subject may be experiencing a cardiac or respiratory condition. Under at least one such circumstance, a tertiary optical sensor measurement may be warranted. The processor 111 may then conditionally process optical signals from the tertiary sensor to generate a more confident assessment of the cardiac or respiratory condition for the subject. For example, for the case where the tertiary sensor 110c is an imaging sensor directed towards the skin (or other perfuse tissue) of the subject, the processor may process the PPG signals to determine if there is an anomaly in the RR-intervals or blood flow dynamics for the subject. If such an anomaly exists, signals from the imaging sensor may be conditionally processed to evaluate the motion of blood vessels over time. The motion of these blood vessels may be related to the subject's blood pressure and blood flow dynamics, and thus processing these signals in context of a physiological model relating PPG data and blood vessel imaging data to a pathology of the heart and/or lungs (i.e., a cardiopulmonary pathology) can be used to assess the cardiac or respiratory condition of the subject.

In a similar form of smart sensing using the biometric waveform analysis system 10, the generation of physiological assessments may be achieved by smart sensing via contingent activation of a plurality of sensors 110. The method may comprise a method of generating a physiological assessment for a subject via a sensor device (e.g., 1200, FIGS. 12A-12B; 1100, FIG. 12C) worn by the subject, wherein the sensor device comprises a motion sensor 110a, a photoplethysmography (PPG) sensor 110b, a tertiary sensor 110c, and at least one processor 111, the method comprising: 1) screening, via the at least one processor 111, a motion signal generated by the motion sensor 110a to determine if PPG monitoring of the subject is warranted, 2) in response to determining that PPG monitoring of the subject is warranted, screening, via the at least one processor 111, a PPG signal from the PPG sensor 110b to determine if monitoring of the subject via the tertiary sensor 110c is warranted, 3) in response to determining that monitoring of the subject via the tertiary sensor 110c is warranted, activating the tertiary sensor 110c via the at least one processor 111, and 4) processing, via the at least one processor 111, the motion signal, the PPG signal, and a signal from the tertiary sensor 110c to generate a physiological assessment for the subject. As described for smart sensing using selective signal processing, smart sensing by selective sensor activation can save power (thereby increasing battery life of a wearable device) and improve subject convenience when wearing the biometric device. It should be noted that this process of smart sensing may be cascaded beyond tertiary sensing to include additional conditional steps for additional sensing (quaternary (4^{th}) quinary (5^{th}), and so on).

This invention is meant to save power resources by contingent activation of sensors and/or contingent signal processing of sensors. The processing, assessment generation, and general methodology of smart sensing by contingent signal processing (processing sensor data only when warranted) and contingent activation (activating sensors only when warranted) may generally be described interchangeably throughout the various embodiments described herein for smart sensing. For example, as with the prior example, in one embodiment, the tertiary sensor is an ECG sensor, and activating the tertiary sensor comprises activating the ECG sensor; similarly, in one embodiment, the tertiary sensor is an auscultatory sensor, and activating the tertiary sensor comprises activating the auscultatory sensor; and so forth. However, in contrast with contingent signal processing, contingent activation implies conditionally powering a sensor on or activating a sensor to an active state when warranted. Thus, contingent sensor activation may also imply contingent signal processing, but contingent signal processing does not necessarily demand contingent signal activation. Namely, for the case of contingent signal processing, although the processing of sensor data may be selectively turned on or off for one or more sensors, all of the sensors themselves may be continuously activated (i.e., be continuously in the “on” mode).

Depending on the sensor required for a particular assessment, smart sensing by contingent sensor activation may be required for the practicality of the use case. For example, in one embodiment, the tertiary sensor may be an optical sensor, such as an imaging sensor (i.e., a camera, optical sensor array, or the like) or a spectrometer (such as an optical spectrometer, Raman spectrometer, or the like). In such case, the high-power draw of these optical sensors may be too high to have these sensors activated continuously. Thus, contingent signal processing (i.e., with continuous sensor activation) may not be enough for power savings, contingent activation of the tertiary sensor may be critical.

For both contingent sensor signal processing and contingent sensor activation, one aforementioned key utility of processing the motion sensor signals was described as limiting the need to activate or process signals from the PPG and tertiary signals by determining if motion artifacts would be low enough to make these sensor readings meaningful. However, for some physiological assessments, the act of moving may be fundamentally critical for the assessments. For example, in one embodiment of smart sensing, the processor may process motion sensor signals to determine that the subject is moving in a suitable manner (such as moving at a steady pace, moving with certain level of intensity, reading or speaking effectively, executing a particular activity, or the like) such that a physiological assessment is achievable. This determination may then warrant the processing of PPG signals to determine the physical activity induced blood flow dynamics for the subject. For example, the change in heart rate with physical activity changes may be processed to determine heart rate recovery dynamics. In addition, if the physical activity induced blood flow dynamics are found to be abnormal or undesirable, the processor may determine that activating (and/or processing data from) a tertiary sensor may be warranted. In this manner, a subject can be effectively monitored (at a lower power budget) to determine if their physiological response is appropriate for the desired activity, and further assessments about their physiological condition may be generated.

Though the biometric waveform analysis system 10 described herein has been described primarily for noninvasive wearable devices, in principle the methods described may also be used for implantable devices (i.e., invasive or minimally invasive biometric devices). In such case, the methods used for biometric monitoring and assessment generation may be identical, but the sensor configurations may be notably different to adhere to biocompatibility requirements and physiological differences associated with implantable devices. Moreover, the types of sensors used for implantable devices may be different (such as biochemical sensors being more particularly useful for implantable devices).

### i) Improving the Accuracy of a Biometric or Physiological Assessment

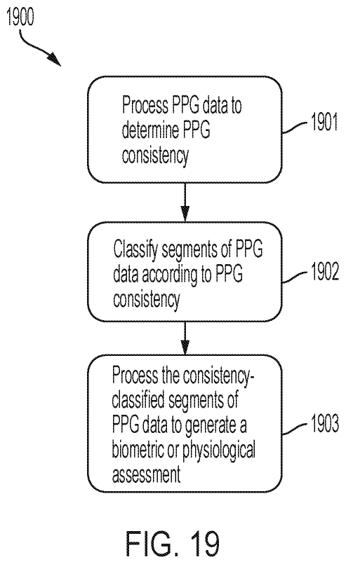

The biometric waveform analysis system 10 enables a system and method for improving the accuracy of a biometric or a physiological assessment via processing data from a PPG sensor. For example, referring to FIG. 19, a method 1900 comprises processing PPG data to determine PPG consistency (Block 1901), e.g., via the data contextualizer 310 or other processor, classifying segments of PPG data according to PPG consistency (Block 1902), e.g., via the waveform classifier 320 or other processor, and processing the consistency-classified segments of PPG data to generate a biometric or physiological assessment (Block 1903), e.g., via the assessment processor 340 or other processor. A key benefit of this novel method 1900 is that the accuracy of a biometric or physiological assessment may be improved by selectively processing PPG data that is classified as having a high consistency (e.g., a high morphological uniformity between a set of temporally adjacent PPG waveforms). Although a variety of physiological assessments may be improved for accuracy via this method 1900, a specific example for improving the accuracy of PPG-based blood pressure is provided below.

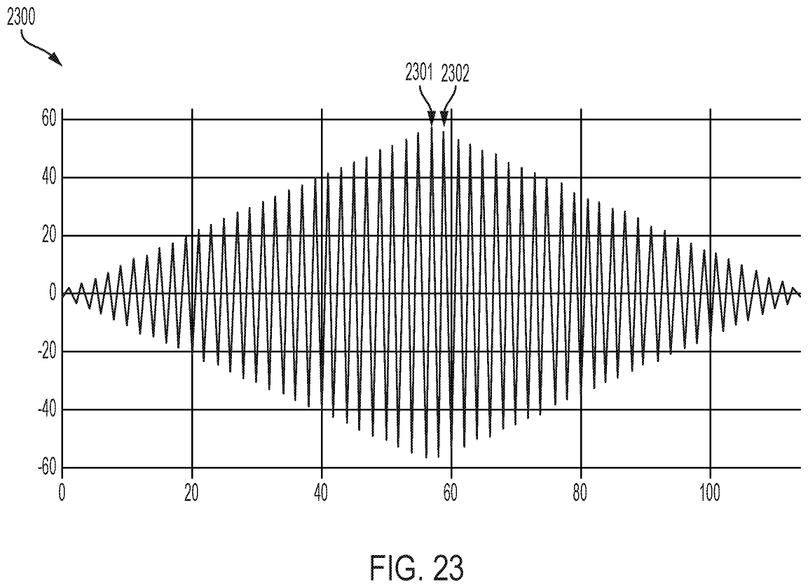

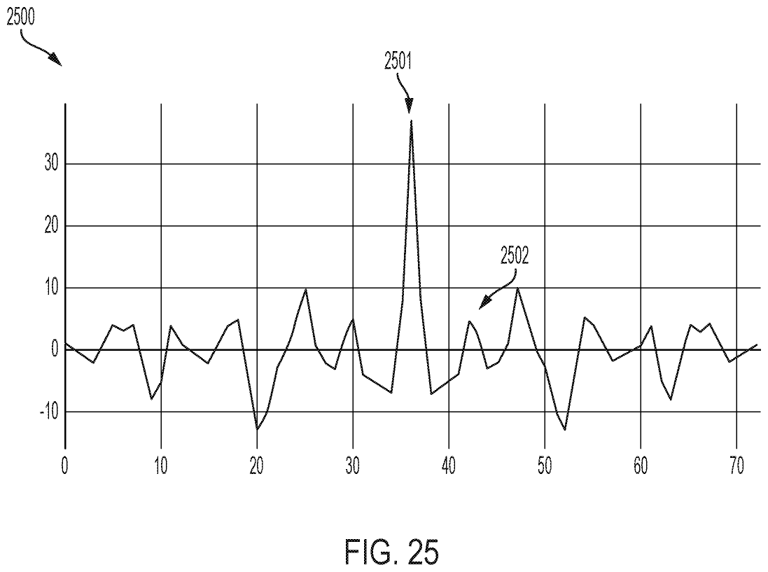

A specific embodiment 2100 of the method 1900 is presented in FIG. 21. The method 2100 comprises processing PPG data to determine PPG consistency via RPR (“relative peak ratio”) (Block 2101), classifying segments of PPG data according to PPG consistency (Block 2102), as determined by RPR, and processing the RPR-classified segments of PPG data to generate a biometric or physiological assessment (Block 2103). The term RPR is used to describe a ratio of the second peak in an autocorrelation (of temporally neighboring PPG waveforms) to the first peak. The higher the RPR for a set of PPG waveforms, the higher the consistency of those waveforms. A graphical example of RPR is presented in the autocorrelation plot 2300 of FIG. 23, with RPR being the ratio of the magnitude of peak 2302 to peak 2301. It will be appreciated that the RPR of FIG. 23 is notably higher than that of FIG. 25 (i.e., the ratio 2302/2301 is notably higher than 2502/2501). It will be appreciated that the RPR value can never be greater than 1.

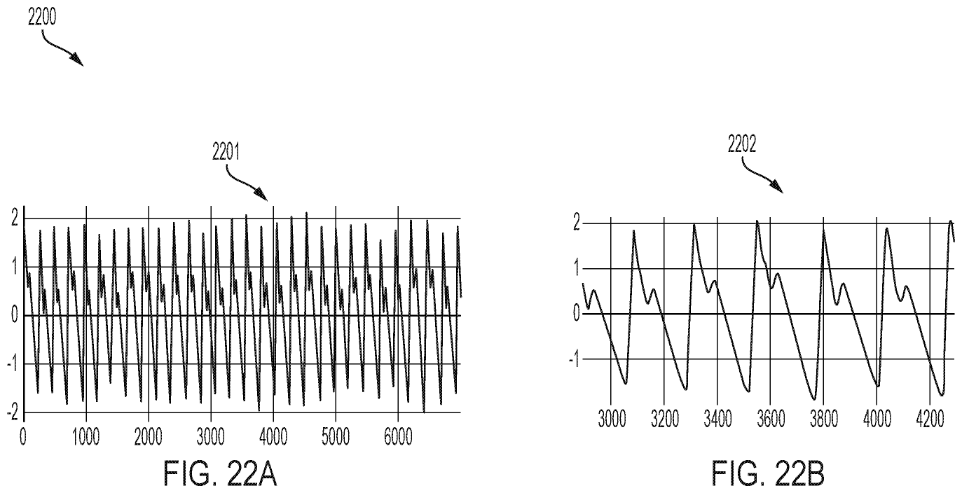

FIG. 23 comprises a graphical representation 2300 of an autocorrelation of a set of PPG waveforms 2100 (FIGS. 22A-22B) having a relatively high consistency (high-RPR), and FIG. 25 comprises a graphical representation 2500 of an autocorrelation of a set of PPG waveforms 2300 (FIGS. 24A-24B) having a relatively low consistency (low-RPR). The high-consistency set of PPG waveforms 2200 is presented in two forms, over a broad range 2201 (FIG. 22A) of the set of waveforms 2200 and over a narrowed range 2202 (FIG. 22B) of the set of waveforms 2200. The low-consistency set of PPG waveforms 2400 (FIGS. 24A-24B) is presented in the same manner, over a broad range 2401 (FIG. 24A) of the set of waveforms 2400 and over a narrowed range 2402 (FIG. 24B) of the set of waveforms 2400. The low consistency of the set of waveforms 2400 in FIGS. 24A-24B may be due to respiration-induced changes in the PPG waveform or other mechanisms which may modulate the blood pressure or PPG uniformity for the subject in time.

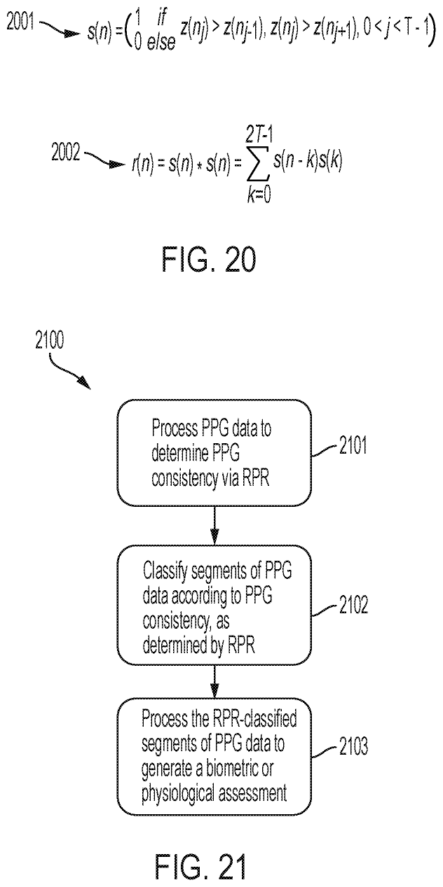

It should be noted that the autocorrelation representations 2300 and 2500 were calculated in the same manner, as summarized by the equations 2001 and 2002 of FIG. 20. In this case, a PPG signal p(t) was periodically sampled at t=n seconds, where n is an integer>0, uniformly over a period T. p(n) was processed by attenuating the DC content and high frequency noise, via a DC-blocking filter and a low-pass filter (LPF), resulting in a processed signal z(n). A time-domain version of a sub-band peak energy detection (SPED) algorithm was then applied to z(n) to generate an adaptive peak normalization across z(n), yielding s(n). The equation 2001 for generating s(n), as well as the autocorrelation equation 2002 used to generate plots 2300 and 2500 are presented in FIG. 20.

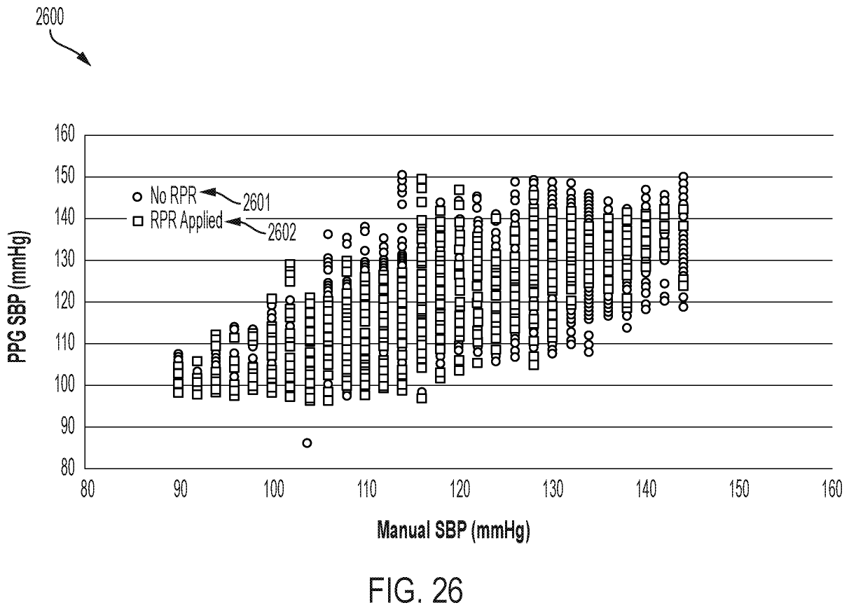

Once PPG waveforms have been classified as “high-RPR” or “low-RPR”, the biometric waveform analysis system 10 may be utilized to generate a physiological assessment based primarily (or solely) upon “high-RPR” PPG waveforms. In a specific example, the method 2100 of FIG. 21 was applied towards hundreds of PPG datasets collected from hundreds of participants, wherein time-correlated sphygmomanometer (manual blood pressure) data was also collected. Each PPG dataset comprised several seconds of PPG waveforms, and each PPG dataset was classified according to a RPR value. A machine learning model had been previously developed, with PPG-based features (such as features from PPG-based metrics, PPG waveforms, and transforms of the PPG waveforms) and static biometric parameters (age, gender, height, and weight) as inputs and a blood pressure estimation (systolic and diastolic) as the output. In this model, one BP estimation for each PPG dataset was generated. An exemplary machine learning model for PPG-based blood pressure is presented in U.S. Pat. No. 10,206,627, which is incorporated herein by reference in its entirety. The model was applied towards the PPG datasets for each subject, along with static biometric parameters (age, gender, height, and weight), to estimate subject blood pressure. A comparison of the estimated (PPG-based) and measured (sphygmomanometer-based) blood pressure values was generated and is presented in the plot 2600 of FIG. 26. There are two results presented in this plot: the PPG-based estimation of blood pressure without RPR 2601 and the PPG-based estimation of blood pressure with RPR applied 2602. The blood pressure estimations without RPR 2601 were generated regardless of the RPR value; in contrast, the blood pressure estimations with RPR applied were filtered by RPR, such that BP estimations were generated only if RPR was determined to be sufficiently high (in this particular case, an RPR>0.4444). It will be appreciated from FIG. 26 that the blood pressure estimations with RPR applied 2602 are notably more accurate than those with no RPR 2601 applied. Indeed, the standard deviation of errors between PPG estimations and sphygmomanometer measurements were found to be more than ±1 mm Hg higher for no RPR vs. RPR applied. For RPR applied 2602, the standard deviation of errors falls below ±8 mm Hg, which is significant for medical relevance. Depending on the PPG datasets analyzed, the applicant has discovered that an RPR>0.4444 may reduce the standard deviation of errors between PPG estimations and sphygmomanometer measurements by more than ±3 mmHg.

It should be noted that alternative methods of generating s(n) and/or the autocorrelation described above may be used, as several methods of generating autocorrelations are well known to those skilled in the art. Moreover, alternative methods of determining the consistency of PPG waveforms other than RPR may be used. For example, rather than a time-domain approach, a rolling spectrogram (windowed over several seconds) of a PPG signal may be generated, yielding several spectrograms associated with temporally contiguous multi-second blocks of data. These spectrograms may then be autocorrelated to assess consistency (e.g., by performing RPR on the autocorrelation of the contiguous spectrograms, or by some other consistency determination method). In such case, an RPR (or the equivalent) may be processed for the autocorrelation of the spectrograms. Additionally, for either temporal or spectral autocorrelations, a ratio of different peaks other than the 2^{nd}/1^{st} peak (e.g., of the autocorrelation plots of FIG. 23 and FIG. 25) may be used in place of RPR. For example, the ratio of the 3^{rd} to 1^{st} peak may be utilized to gauge consistency, or a combination of multiple peak ratios may be utilized. Alternatively, a pattern associated with the autocorrelation plots may be utilized to gauge consistency. For example, a diamond shape of FIG. 23 may be indicative of a higher consistency than an erratic shape of FIG. 25. Thus, rather than, or in addition to, an RPR analysis, the waveform analysis engine 300 may be configured to identify a pattern (such as a diamond pattern, an erratic pattern, or the like) in the autocorrelation of the waveforms that is indicative of high or low waveform consistency. Thus, in short, while the RPR method detailed herein has been proven to be quite effective in improving the accuracy of blood pressure estimations, a variety of methods may be used to assess waveform consistency, and the important theme is that the consistency of the temporally contiguous PPG waveforms should be assessed and scored in consistent manner.

It should be noted that, although the method 1900 (FIG. 19), and also the subset method 2100 (FIG. 21), is directed towards PPG waveforms, other physiological waveforms may also be utilized in this invention to improve the accuracy of a physiological assessment. For example, the methods 1900 and 2100 may alternatively be executed by replacing “PPG” with ECG, EEG, EOG, EMG, or other physiological waveforms. In a different embodiment, the autocorrelation 2002 may be substituted with a convolution or a correlation between two different physiological waveforms. For example, a PPG waveform may be convolved with a time-correlated ECG waveform in order to gauge the consistency of disparate (physiologically distinct) waveforms in comparison with each other (as opposed to the consistency of a set of PPG waveforms against itself). This type of consistency analysis, between different waveforms, may be particularly useful when making an analysis to gauge user intent. For example, the consistency between time-correlated EEG waveforms and PPG waveforms may be indicative of an intent to take action (e.g., as with the intent to make a body motion).

Example embodiments are described herein with reference to block diagrams and flow diagrams. It is understood that a block of the block diagrams and flow diagrams, and combinations of blocks in the block diagrams and flow diagrams, can be implemented by computer program instructions that are performed by one or more computer circuits, such as electrical circuits having analog and/or digital elements. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and flow diagrams, and thereby create means (functionality) and/or structure for implementing the functions/acts specified in the block diagrams and flow diagrams.

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and flow diagrams.

A tangible, non-transitory computer-readable medium may include an electronic, magnetic, optical, electromagnetic, or semiconductor data storage system, apparatus, or device. More specific examples of the computer-readable medium would include the following: a portable computer diskette, a random access memory (RAM) circuit, a read-only memory (ROM) circuit, an erasable programmable read-only memory (EPROM or Flash memory) circuit, a portable compact disc read-only memory (CD-ROM), and a portable digital video disc read-only memory (DVD/BlueRay).

The computer program instructions may also be loaded onto a computer and/or other programmable data processing apparatus to cause a series of operational steps to be performed on the computer and/or other programmable apparatus to produce a computer-implemented process such that the instructions which execute on the computer or other programmable apparatus provide steps for implementing the functions/acts specified in the block diagrams and flow diagrams. Accordingly, embodiments of the present invention may be embodied in hardware and/or in software (including firmware, resident software, micro-code, etc.) that runs on a processor such as a digital signal processor, which may collectively be referred to as “logic”, “circuitry,” “a module”, “an engine” or variants thereof.

It should also be noted that the functionality of a given block of the block diagrams and flow diagrams may be separated into multiple blocks and/or the functionality of two or more blocks of the block diagrams and flow diagrams may be at least partially integrated. Finally, other blocks may be added/inserted between the blocks that are illustrated. Moreover, although some of the diagrams include arrows on communication paths to show a primary direction of communication, it is to be understood that communication may occur in the opposite direction to the depicted arrows.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

1-68. (canceled)

69. A method of generating a physiological assessment for a subject via a sensor device worn by the subject, wherein the sensor device comprises a motion sensor, a photoplethysmography (PPG) sensor, a tertiary sensor, and at least one processor, the method comprising:
screening, via the at least one processor, a motion signal generated by the motion sensor to determine if PPG monitoring of the subject is warranted;
in response to determining that PPG monitoring of the subject is warranted, screening, via the at least one processor, a PPG signal from the PPG sensor to determine if monitoring of the subject via the tertiary sensor is warranted;
in response to determining that monitoring of the subject via the tertiary sensor is warranted, screening, via the at least one processor, a signal from the tertiary sensor; and
processing, via the at least one processor, the signal from the tertiary sensor to generate a physiological assessment for the subject.

70. The method of claim 69, wherein the tertiary sensor is an ECG sensor and the tertiary signal is an ECG signal.

71. The method of claim 69, wherein the tertiary sensor is an acoustic sensor and the tertiary signal is an auscultatory signal.

72. The method of claim 70, wherein the physiological assessment comprises an assessment of a cardiac condition of the subject.

73. The method of claim 70, wherein the physiological assessment comprises an assessment of a presence of arrhythmia in the subject.

74. The method of claim 71, wherein the physiological assessment comprises an assessment of a breathing condition of the subject.

75. The method of claim 70, wherein the physiological assessment comprises an assessment that a cardiac condition is imminent.

76. The method of claim 71, wherein the physiological assessment comprises an assessment that a respiratory condition is imminent.

77. A method of generating a physiological assessment for a subject via a sensor device worn by the subject, wherein the sensor device comprises a motion sensor, a photoplethysmography (PPG) sensor, a tertiary sensor, and at least one processor, the method comprising:
screening, via the at least one processor, a motion signal generated by the motion sensor to determine if PPG monitoring of the subject is warranted;
in response to determining that PPG monitoring of the subject is warranted, screening, via the at least one processor, a PPG signal from the PPG sensor to determine if monitoring of the subject via the tertiary sensor is warranted;
in response to determining that monitoring of the subject via the tertiary sensor is warranted, activating the tertiary sensor via the at least one processor; and
processing, via the at least one processor, the motion signal, the PPG signal, and a signal from the tertiary sensor to generate a physiological assessment for the subject.

78. The method of claim 77, wherein the tertiary sensor is an ECG sensor, and wherein activating the tertiary sensor comprises activating the ECG sensor.

79. The method of claim 77, wherein the tertiary sensor is an auscultatory sensor, and wherein activating the tertiary sensor comprises activating the auscultatory sensor.

80. The method of claim 77, wherein the tertiary sensor is an optical sensor, and wherein activating the tertiary sensor comprises activating the optical sensor to emit light into the body of the subject.

81. The method of claim 77, wherein the physiological assessment comprises an assessment of heart rate variably of the subject.

82. The method of claim 77, wherein the tertiary sensor is a camera sensor, and wherein activating the tertiary sensor comprises activating the camera.

83. The method of claim 77, wherein the physiological assessment comprises an assessment of a cardiac or respiratory condition of the subject.

84. A method of generating a physiological assessment of a subject, the method comprising:
obtaining a photoplethysmography (PPG) data signal from a PPG sensor worn on a body of the subject, wherein the PPG data signal comprises a plurality of PPG waveforms;
processing the PPG data signal to determine consistency of the PPG waveforms;
classifying the PPG waveforms according to waveform consistency; and
preferentially processing the PPG waveforms having a classification of higher consistency to generate a physiological assessment for the subject, such that the physiological assessment is more accurate than if all PPG waveforms had been utilized.

85. The method of claim 84, wherein determining the consistency of the PPG waveforms comprises determining a relative-peak-ratio (RPR) for the PPG waveforms.

86. The method of claim 84, wherein the physiological assessment comprises an assessment of subject blood pressure.

87. The method of claim 85, wherein determining relative-peak-ratio (RPR) comprises determining a ratio of a second peak to a first peak in an autocorrelation of temporally neighboring PPG waveforms.

88. The method of claim 85, wherein determining relative-peak-ratio (RPR) comprises determining a ratio of a third peak to a first peak in an autocorrelation of temporally neighboring PPG waveforms.
